(19) United States
(12) Patent Application Publication
(10) Pub. No.: US 2026/0022388 A1
(43) Pub. Date: Jan. 22, 2026

(54) SYSTEMS FOR CELL PROGRAMMING AND METHODS THEREOF

(71) Applicant: Syntax Bio, Inc., Chicago, IL (US)

(72) Inventors: Ryan CLARKE, Chicago, IL (US); Bradley J. MERRILL, Oak Park, IL (US); Matthew MACDOUGALL, Menlo Park, CA (US); Nikolas George Koutis BALANIS, Chicago, IL (US); Renee DE POOTER, Chicago, IL (US); Caspian HARDING, Chicago, IL (US); Georgina MANCINELLI, Chicago, IL (US)

(21) Appl. No.: 18/806,125

(22) Filed: Aug. 15, 2024

Related U.S. Application Data
(63) Continuation of application No. PCT/US23/13240, filed on Feb. 16, 2023.
(60) Provisional application No. 63/311,122, filed on Feb. 17, 2022.

(51) Int. Cl. C12N 15/63; C12N 9/22; C12N 15/10; C12N 15/113; C12N 15/64; C12N 15/85

(52) U.S. Cl. CPC C12N 15/635; C12N 9/22; C12N 15/10; C12N 15/113; C12N 15/64; C12N 15/85; C12N 2510/00

(57) ABSTRACT

Provided herein are systems of modulating gene expression, methods of use thereof, and cells engineered thereof for the purpose of differentiating cells, for example immune cells.

### CROSS REFERENCE

This application is a continuation of International Application No. PCT/US23/13240, filed Feb. 16, 2023, which claims the benefit of U.S. Provisional Patent Application No. 63/311,122, filed on Feb. 17, 2022, each of which is incorporated herein by reference in its entirety.

### BACKGROUND

Heterologous proteins and/or nucleic acid molecules can be utilized to elicit a desired response in a cell. The heterologous proteins and/or nucleic acid molecules can regulate genes of interest (e.g., transgenes and/or endogenous genes) to program (e.g., differentiate, de-differentiate) a cell. In some cases, endonuclease-based technologies (e.g., clustered regularly interspaced short palindromic repeats (CRISPR)-associated protein or “CRISPR/Cas”) have been adopted for manipulation of polynucleotide sequences, epigenetic modification thereof, and/or expression level thereof. For example, the CRISPR/Cas technology can be characterized by its versatility and facile programmability and can be used to promote genome editing across different species.

### SUMMARY

The present disclosure provides methods for inducing a desired expression and/or activity profile of a target gene in a cell, the method comprising: contacting the cell with a heterologous genetic circuit comprising a plurality of gate units, wherein, upon activation of the heterologous genetic circuit, the plurality of gate units operates in concert to induce a plurality of distinct modulations of the target gene in a sequential manner, each of the plurality of distinct modulations being necessary but individually insufficient to effect the desired expression and/or activity profile of the target gene, wherein the plurality of gate units comprises: (i) a first gate unit that is activatable, upon the activation of the heterologous genetic circuit, to induce a first distinct modulation of the plurality of distinct modulations; and (ii) a second gate unit that is activatable upon the activation of the heterologous genetic circuit, to induce a second distinct modulation of the plurality of distinct modulations, wherein the second distinct modulation is induced subsequent to the first distinct modulation, such that the first distinct modulation and the second distinct modulation both enhance or both reduce expression and/or activity level of the target gene in the cell, wherein, upon the contacting, the plurality of gate units operates in concert to effect the desired expression and/or activity profile of the target gene in the cell.

The present disclosure also provides systems for inducing a desired expression and/or activity profile of a target gene in a cell, the system comprising: a heterologous genetic circuit comprising a plurality of gate units, wherein, upon activation of the heterologous genetic circuit, the plurality of gate units operates in concert to induce a plurality of distinct modulations of the target gene in a sequential manner, each of the plurality of distinct modulations being necessary but individually insufficient to effect the desired expression and/or activity profile of the target gene, wherein the plurality of gate units comprises: (i) a first gate unit that is activatable, upon the activation of the heterologous genetic circuit, to induce a first distinct modulation of the plurality of distinct modulations; and (ii) a second gate unit that is activatable upon the activation of the heterologous genetic circuit, to induce a second distinct modulation of the plurality of distinct modulations, wherein the second distinct modulation is induced subsequent to the first distinct modulation, such that the first distinct modulation and the second distinct modulation both enhance or both reduce expression and/or activity level of the target gene in the cell, wherein, upon the activation of the heterologous genetic circuit, the plurality of gate units operates in concert to effect the desired expression and/or activity profile of the target gene in the cell.

The present disclosure also provides methods for inducing a desired expression and/or activity profile of a target gene in a cell, the method comprising: contacting the cell with a heterologous genetic circuit comprising a plurality of gate units, wherein, upon activation of the heterologous genetic circuit, the plurality of gate units operates in concert to induce a plurality of distinct modulations of the target gene in a sequential manner, each of the plurality of distinct modulations being necessary but individually insufficient to effect the desired expression and/or activity profile of the target gene, wherein the plurality of gate units comprises: (i) a first gate unit that is activatable, upon the activation of the heterologous genetic circuit, to induce a first distinct modulation of the plurality of distinct modulations; and (ii) a second gate unit that is activatable to induce disruption of the first gate unit that has been activated, wherein the inactivation induces a second distinct modulation of the plurality of distinct modulations, wherein the second distinct modulation is induced subsequent to the first distinct modulation, such that the second distinct modulation attenuates the first distinct modulation, wherein, upon the contacting, the plurality of gate units operates in concert to effect the desired expression and/or activity profile of the target gene in the cell.

The present disclosure also provides systems for inducing a desired expression and/or activity profile of a target gene in a cell, the system comprising: a heterologous genetic circuit comprising a plurality of gate units, wherein, upon activation of the heterologous genetic circuit, the plurality of gate units operates in concert to induce a plurality of distinct modulations of the target gene in a sequential manner, each of the plurality of distinct modulations being necessary but individually insufficient to effect the desired expression and/or activity profile of the target gene, wherein the plurality of gate units comprises: (i) a first gate unit that is activatable, upon the activation of the heterologous genetic circuit, to induce a first distinct modulation of the plurality of distinct modulations; and (ii) a second gate unit that is activatable to induce disruption of the first gate unit that has been activated, wherein the inactivation induces a second distinct modulation of the plurality of distinct modulations, wherein the second distinct modulation is induced subsequent to the first distinct modulation, such that the second distinct modulation attenuates the first distinct modulation, wherein, upon the activation of the heterologous genetic circuit, the plurality of gate units operates in concert to effect the desired expression and/or activity profile of the target gene in the cell.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows the autologous T-cell development pathways in a circuit model.

FIG. 2 shows the genes known to affect T-cell differentiation at different steps during T-cell development.

FIG. 3 depicts how the heterologous genetic circuits (HGC) function to further T-cell development at different stages as compared to the theorized functions of autologous T-cell development pathways.

FIG. 4 illustrates guide nucleic acid molecules (e.g., gRNAs) in accordance with the systems and methods of the present disclosure.

FIGS. 5A-5I illustrate examples of heterologous genetic circuits (HGC) in accordance with the systems and methods of the present disclosure. FIG. 5A depicts T-Cell HGC1. FIG. 5B depicts T-Cell HGC2. FIG. 5C depicts T-Cell HGC3. FIG. 5D depicts T-Cell HGC4. FIG. 5E depicts T-Cell HGC5. FIG. 5F depicts T-Cell HGC6. FIG. 5G depicts T-Cell HGC7. FIG. 5H depicts T-Cell HGC8. FIG. 5I depicts T-Cell HGC9.

FIG. 6 depicts the intermediate T-cell stages induced by the HGCs overlaid onto a traditional T-cell development pathway, used for reference.

FIG. 7 is a schematic of the heterologous genetic circuit. An activating moiety initiates the circuit and can activate a gate unit. A gate unit is comprised of a gate moiety and/or a gene regulating moiety.

FIG. 8 depicts exemplary heterologous genetic circuits.

FIG. 9 shows scatter plots (i.e., a volcano plots) at days 3, 5, and 9 to identify one or more heterologous genetic circuits that differentiated induced pluripotent stem cells (iPSCs) into hematopoietic progenitor cells.

FIGS. 10A-10D show examples of hematopoietic progenitor cell marker analysis data used to generate the scatter plots of FIG. 9. FIG. 10A shows results for cell marker CD34⁺. FIG. 10B shows results for cell marker CD43⁺. FIG. 10C shows results for cell marker CD45⁺. FIG. 10D shows results for the combination of cell markers CD34⁺/CD43⁻/CD45⁺. The left and right dots represent replicate samples for each condition. The center dot represents mean value.

FIGS. 11A-11D show that the heterologous genetic circuits generate substantially more hematopoietic progenitor cells after five days in culture as compared to directed differentiation. FIG. 11A shows cell marker results for CD34⁺ and CD45⁺ markers. FIG. 11B shows additional cell marker results for CD34⁺, CD43⁺, and CD45⁺ markers. FIG. 11C shows a summary of the data of FIG. 11B. FIG. 11D shows a summary of the results from a repeat experiment.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used in the specification and claims, the singular forms “a,” “an,” and “the” include plural references unless the context clearly dictates otherwise. For example, the term “a gate unit” includes a plurality of gate units.

The term “about” or “approximately” generally mean within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated, the term “about” meaning within an acceptable error range for the particular value should be assumed.

The use of the alternative (e.g., “or”) should be understood to mean either one, both, or any combination thereof of the alternatives. The term “and/or” should be understood to mean either one, or both of the alternatives.

### Definitions

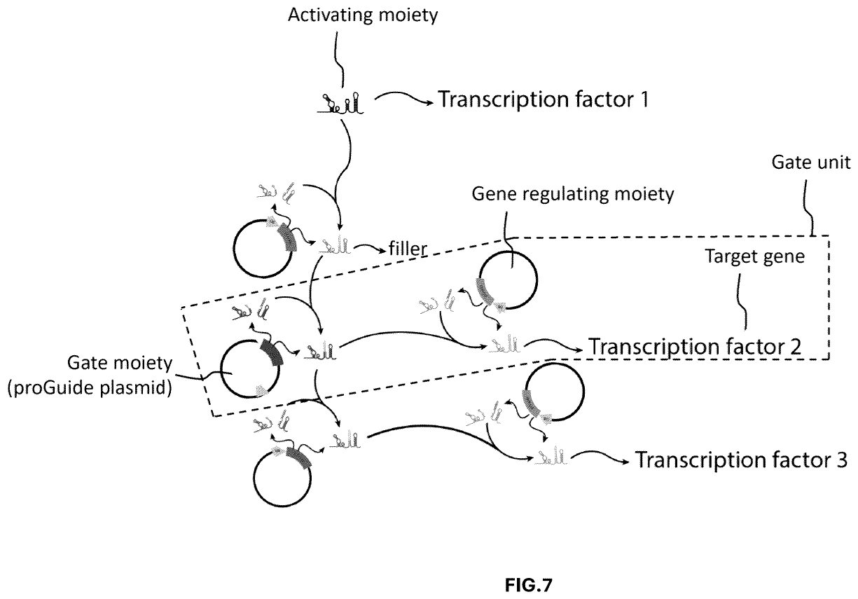

The term “genetic circuit,” “biological circuit,” or “circuit,” as used interchangeably herein, generally refers to a collection of molecular components (e.g., biological materials, such as polypeptides and/or polynucleotides, non-biological materials, etc.) operatively coupled (e.g., operating simultaneously, sequentially, etc.) accordingly to a circuit design. The collection of the molecular components can be capable of providing one or more specific outputs in a cell (e.g., regulation of one or more genes) in response to one or more inputs (e.g., a single input or a plurality of inputs). Such one or more inputs can be sufficient to trigger the molecular components of the genetic circuit to provide the one or more specific outputs. For example, the genetic circuit can comprise one or more molecular switches that are activatable by one or more inputs (FIG. 7).

A genetic circuit can be a controllable gene expression system comprising an assembly of biological parts that work together (e.g., simultaneously, sequentially, etc.) as a logical function. A genetic circuit can comprise a plurality of gate units, wherein at least one gate unit of the plurality of gate units is activatable by an activating moiety (e.g., a heterologous input to the cell) to activate other gate units of the plurality of gate units (e.g., simultaneously at once, sequentially in a cascading manner, etc.) (FIG. 7). For example, at least one gate unit of the plurality of gate units can be activatable (e.g., directly or indirectly) by another gate unit of the plurality of gate units, to (i) regulate expression or activity level of one or more target genes, (ii) activate at least one another gate unit of the plurality of gate units, and/or (ii) deactivate at least one another gate unit of the plurality of gate units, thereby collectively regulating expression and/or activity level of one or more target genes in a desired manner, as predetermined by the design of the genetic circuit (FIG. 7). The terms “heterologous genetic circuit,” “HGC,” “cellular algorithm,” or “cellgorithm” as used herein may be used interchangeably.

The term “gate unit,” as referred to herein, generally refers to a portion of the genetic circuit that can control gene regulation by functioning similarly to a logic gate wherein it can control the flow of information and allow the circuit to multiplex decision making at different points. More specifically, the term refers to a nucleic acid encoding a genetic switch and a transcription/translation regulatory region, or series of regions, which the genetic switch acts on. The input for a gate unit can be an activating moiety and/or another gate unit. The output for a gate unit can be used to activate another gate unit, to de-activate another gate unit, to affect a target gene, and/or a combination of any of the above. For example, a gate unit can be comprised of a plurality of gate moieties and/or a plurality of gene regulating moieties (FIG. 7).

The term “activating moiety,” as referred to herein, generally refers to a moiety that can activate plurality of genetic circuits and/or a plurality of gate units. An activating moiety can be a heterologous input to a cell. In some cases, activating moieties can include, but are not limited to, a guide nucleic acid molecule (e.g., a gRNA) or other nucleic acid, polypeptides, polynucleotides, small molecules, light, or a combination thereof. For example, an activating moiety can be a guide nucleic acid molecule that forms a complex with an endonuclease (e.g., a Cas protein) to bind to a polynucleotide sequence of a gate moiety (e.g., a plasmid encoding another guide nucleic acid molecule) that is inactivated, to activate such gate moiety (e.g., induce expression of a functional form of the additional guide nucleic acid molecule) that can target one or more gene regulating moieties.

The term “gate moiety,” as referred to herein, generally refers to a moiety that can affect the function of a gene regulating moiety within a gate unit. A gate moiety can activate and/or deactivate a gene regulating moiety. For example, a gate moiety can regulate expression of a gene regulation moiety by editing a nucleic acid sequence and thereby activating or deactivating the gene regulating moiety. For example, a gate moiety can be a guide nucleic acid molecule that forms a complex with an endonuclease (e.g., a Cas protein) to bind to a polynucleotide sequence of a gene regulating moiety (e.g., a plasmid encoding another guide nucleic acid molecule) to activate the gene regulating moiety (e.g., induce expression of a functional form of the another guide nucleic acid molecule) that can target one or more endogenous genes of a cell. Alternatively or in addition to, a gate moiety can activate and/or deactivate another gate unit of the genetic circuit (FIG. 7). For example, a gate moiety can be a guide nucleic acid molecule that forms a complex with an endonuclease (e.g., a Cas protein) to bind to a polynucleotide sequence of another gate moiety (e.g., a plasmid encoding another guide nucleic acid molecule) that is inactivated, to activate the another gate moiety (e.g., induce expression of a functional form of the another guide nucleic acid molecule). In another example, a gate moiety can be a guide nucleic acid molecule that forms a complex with an endonuclease (e.g., a Cas protein) to bind to a polynucleotide sequence of another gate moiety (e.g., a plasmid encoding another guide nucleic acid molecule) that is activated, to inactivate the another gate moiety (e.g., reduce expression of a functional form of the another guide nucleic acid molecule).

The term “gene regulating moiety” or “gene editing moiety” as used interchangeably herein, generally refers to a moiety which can regulate the expression and or activity profile of a nucleic acid sequence or protein, whether exogenous or endogenous to a cell (FIG. 7). For example, a gene editing moiety can regulate expression of a gene by editing a nucleic acid sequence (e.g. CRISPR-Cas, Zinc-finger nucleases, TALENs, or siRNA). In some cases, a gene editing moiety can regulate expression of a gene by editing a genomic DNA sequence. In some cases, a gene editing moiety can regulate expression of a gene by editing an mRNA template. Editing a nucleic acid sequence can, in some cases, alter the underlying template for gene expression (e.g. CRISPR-Cas-inspired RNA targeting systems). Alternatively, a gene editing moiety can repress translation of a gene (e.g. Cas13).

Alternatively or in addition to, a gene editing moiety can be capable of regulating expression or activity of a gene by specifically binding to a target sequence operatively coupled to the gene (or a target sequence within the gene), and regulating the production of mRNA from DNA, such as chromosomal DNA or cDNA. For example, a gene editing moiety can recruit or comprise at least one transcription factor that binds to a specific DNA sequence, thereby controlling the rate of transcription of genetic information from DNA to mRNA. A gene editing moiety can itself bind to DNA and regulate transcription by physical obstruction, for example preventing proteins such as RNA polymerase and other associated proteins from assembling on a DNA template. A gene editing moiety can regulate expression of a gene at the translation level, for example, by regulating the production of protein from mRNA template. In some cases, a gene editing moiety can regulate gene expression by affecting the stability of an mRNA transcript. In some cases, a gene editing moiety can regulate a gene through epigenetic editing (e.g. Cas12).

In some cases, a plasmid can encode a non-functional form of a gene editing moiety. The plasmid can be activated to express a functional form of the gene editing moiety, e.g., via activation of a functional gate moiety. For example, the gene editing moiety can encode a non-functional form of a guide nucleic acid molecule that would otherwise be able to bind to a target gene of a cell. Upon binding of a functional gate moiety (e.g., another guide nucleic acid molecule complexed with a Cas protein) to the plasmid, the plasmid can be edited to allow expression of a functional form of the gene editing moiety (e.g., a functional form of the guide nucleic acid molecule with specific binding to the target gene of the cell), to permit modulation of the target gene in the cell.

Accordingly, a gene regulating moiety can comprise a nucleic acid molecule (e.g., a guide nucleic acid molecule that forms a complex with an endonuclease, such as a Cas protein). Alternatively or in addition to, a gene regulating moiety can comprise or be operatively coupled to an endonuclease. An endonuclease can be an enzyme that cleaves a phosphodiester bond within a polynucleotide chain. An endonuclease can comprise restriction endonucleases that cleave DNA at specific sites without damaging bases. Restriction endonucleases can include Type I, Type II, Type III, and Type IV endonucleases, which can further include subtypes. In some cases, an endonuclease can be Cas1, Cas2, Cas 3, Cas4, Cas5, Cas6, Cas7, Cas8a, Cas8b, Cas8c, Cas9, Cas10, Cas10d, Cas12, Cas12a (Cpf1), Cas12b (C2c1), Cas12c (C2c3), Cas12d (CasY), Cas12e (CasX), Cas12f (Cas14 or C2c10), Cas12g, Cas12h, Cas12i, Cas12k (C2c5), Cas 13 (C2c2), Cas13b, Cas13c, Cas13d, Cas13x.1, Cse1, Cse2, Csy1, Csy2, Csy3, Csm2, Cmr5, Csx10, Csx11, Csf1, Csn2. An endonuclease can be a dead endonuclease which exhibits reduced cleavage activity. For example, an endonuclease can be a nuclease inactivated Cas such as a dCas (e.g., dCas9).

A gene regulating moiety can be a transcriptional modulator system (e.g., a gene repressor complex or a gene activator complex). For example, a gene regulating moiety can be a gene repressor complex comprising a dCas protein operatively coupled to (e.g., fused to) a transcriptional repressor. Non-limiting examples of transcriptional repressors can include KRAB, SID, MBD2, MBD3, DNMT1, DNMT2A, DNMT3A, DNMT3B, DNMT3L, Mecp2, FOG1, ROM2, LSD1, ERD, SRDX repression domain, Pr-SET7/8, SUV4-20H1, RIZ1, JMJD2A, JHDM3A, JMJD2B, JMJD2C, GASCI, JMJD2D, JARID1A, RBP2, JARIDlB/PLU-1, JARIDIC/SMCX, JARIDID/SMCY, HDAC1, HDAC2, HDAC3, HDAC8, HDAC4, HDAC5, HDAC7, HDAC9, SIRT1, SIRT2, HDACl1, M.Hhal, METI, DRM3, ZMET2, CMT1, CMT2, Lamin A, and Lamin B. Alternatively, a gene regulating moiety can be a gene activator complex comprising a dCas protein operatively coupled to (e.g., fused to) a transcriptional activator. Non-limiting examples of transcriptional activators can include VP16, VP64, VP48, VP160, p65 subdomain, SETIA, SETIB, MLL1, MLL2, MLL3, MLL4, MLL5, ASHI, SYMD2, NSD1, JHDM2a, JHDM2b, UTX, JMJD3, GCN5, PCAF, CBP, p300, TAF1, TIP60/PLIP, MOZ/MYST3, MORF/MYST4, SRCI, ACTR, P160, CLOCK, TETICD, TETI, DME, DML1, DML2, and ROS1.

In some cases, the gene regulating moiety has enzymatic activity that modifies the target gene without cleaving the target gene. Modification of the target gene can cause, for example, epigenetic modifications that can modify gene expression and/or activity level. Examples of enzymatic activity that can be provided by a gene regulating moiety can include but are not limited to: nuclease activity such as that provided by a restriction enzyme (e.g., Fokl nuclease), methyltransferase activity such as that provided by a methyltransferase (e.g., Hhal DNA m5c-methyltransferase (M.Hhal), DNA methyltransferase 1 (DNMT1), DNA methyltransferase 3a (DNMT3a), DNA methyltransferase 3b (DNMT3b), METI, DRM3, ZMET2, CMT1, CMT2; demethylase activity such as that provided by a demethylase (e.g., Ten-Eleven Translocation (TET) dioxygenase 1 (TETICD), TETI, DME, DML1, DML2, ROS 1), DNA repair activity, DNA damage activity, deamination activity such as that provided by a deaminase (e.g., a cytosine deaminase enzyme such as APOBEC1), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity.

Unless specifically stated or obvious from context, the term “polynucleotide,” “oligonucleotide,” or “nucleic acid,” as used interchangeably herein, generally refers to a polymeric form of nucleotides of any length, either deoxyribonucleotides or ribonucleotides, or analogs thereof, either in single-, double-, or multi-stranded form. A polynucleotide can be exogenous or endogenous to a cell. A polynucleotide can exist in a cell-free environment. A polynucleotide can be a gene or fragment thereof. A polynucleotide can be DNA. A polynucleotide can be RNA. A polynucleotide can have any three-dimensional structure, and can perform any function, known or unknown. A polynucleotide can comprise one or more analogs (e.g. altered backbone, sugar, or nucleobase). If present, modifications to the nucleotide structure can be imparted before or after assembly of the polymer. Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g. rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. Non-limiting examples of polynucleotides include coding or non-coding regions of a gene or gene fragment, loci (locus) defined from linkage analysis, exons, introns, messenger RNA (mRNA), transfer RNA (tRNA), ribosomal RNA (rRNA), short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, cell-free polynucleotides including cell-free DNA (cfDNA) and cell-free RNA (cfRNA), nucleic acid probes, and primers. The sequence of nucleotides can be interrupted by non-nucleotide components.

The term “gene” generally refers to a nucleic acid (e.g., DNA such as genomic DNA and cDNA) and its corresponding nucleotide sequence that is involved in encoding an RNA transcript. The term as used herein with reference to genomic DNA includes intervening, non-coding regions as well as regulatory regions and can include 5′ and 3′ ends. In some uses, the term encompasses the transcribed sequences, including 5′ and 3′ untranslated regions (5′-UTR and 3′-UTR), exons and introns. In some genes, the transcribed region will contain “open reading frames” that encode polypeptides. In some uses of the term, a “gene” comprises only the coding sequences (e.g., an “open reading frame” or “coding region”) necessary for encoding a polypeptide. In some cases, genes do not encode a polypeptide, for example, ribosomal RNA genes (rRNA) and transfer RNA (tRNA) genes. In some cases, the term “gene” includes not only the transcribed sequences, but in addition, also includes non-transcribed regions including upstream and downstream regulatory regions, enhancers and promoters. A gene can refer to an “endogenous gene” or a native gene in its natural location in the genome of an organism. A gene can refer to an “exogenous gene” or a non-native gene. A non-native gene can refer to a gene not normally found in the host organism, but which is introduced into the host organism by gene transfer. A non-native gene can also refer to a gene not in its natural location in the genome of an organism. A non-native gene can also refer to a naturally occurring nucleic acid or polypeptide sequence that comprises mutations, insertions and/or deletions (e.g., non-native sequence).

The term “expression” generally refers to one or more processes by which a polynucleotide is transcribed from a DNA template (such as into an mRNA or other RNA transcript) and/or the process by which a transcribed mRNA is subsequently translated into peptides, polypeptides, or proteins. Transcripts and encoded polypeptides can be collectively referred to as “gene product.” If the polynucleotide is derived from genomic DNA, expression can include splicing of the mRNA in a eukaryotic cell. “Up-regulated,” with reference to expression, generally refers to an increased expression level of a polynucleotide (e.g., RNA such as mRNA) and/or polypeptide sequence relative to its expression level in a wild-type state while “down-regulated” generally refers to a decreased expression level of a polynucleotide (e.g., RNA such as mRNA) and/or polypeptide sequence relative to its expression in a wild-type state. Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell.

The term “peptide,” “polypeptide,” or “protein,” as used interchangeably herein, generally refers to a polymer of at least two amino acid residues joined by peptide bond(s). This term does not connote a specific length of polymer, nor is it intended to imply or distinguish whether the peptide is produced using recombinant techniques, chemical or enzymatic synthesis, or is naturally occurring. The terms apply to naturally occurring amino acid polymers as well as amino acid polymers comprising at least one modified amino acid. In some cases, the polymer can be interrupted by non-amino acids. The terms include amino acid chains of any length, including full length proteins, and proteins with or without secondary and/or tertiary structure (e.g., domains). The terms also encompass an amino acid polymer that has been modified, for example, by disulfide bond formation, glycosylation, lipidation, acetylation, phosphorylation, oxidation, and any other manipulation such as conjugation with a labeling component. The terms “amino acid” and “amino acids,” as used herein, generally refer to natural and non-natural amino acids, including, but not limited to, modified amino acids and amino acid analogues. Modified amino acids can include natural amino acids and non-natural amino acids, which have been chemically modified to include a group or a chemical moiety not naturally present on the amino acid. Amino acid analogues can refer to amino acid derivatives. The term “amino acid” includes both D-amino acids and L-amino acids.

The term “derivative,” “variant,” or “fragment,” as used interchangeably herein with reference to a polypeptide, generally refers to a polypeptide related to a wild type polypeptide, for example either by amino acid sequence, structure (e.g., secondary and/or tertiary), activity (e.g., enzymatic activity) and/or function. Derivatives, variants and fragments of a polypeptide can comprise one or more amino acid variations (e.g., mutations, insertions, and deletions), truncations, modifications, or combinations thereof compared to a wild type polypeptide.

The term “engineered,” “chimeric,” or “recombinant,” as used herein with respect to a polypeptide molecule (e.g., a protein), generally refers to a polypeptide molecule having a heterologous amino acid sequence or an altered amino acid sequence as a result of the application of genetic engineering techniques to nucleic acids which encode the polypeptide molecule, as well as cells or organisms which express the polypeptide molecule. The term “engineered” or “recombinant,” as used herein with respect to a polynucleotide molecule (e.g., a DNA or RNA molecule), generally refers to a polynucleotide molecule having a heterologous nucleic acid sequence or an altered nucleic acid sequence as a result of the application of genetic engineering techniques. Genetic engineering techniques include, but are not limited to, PCR and DNA cloning technologies; transfection, transformation and other gene transfer technologies; homologous recombination; site-directed mutagenesis; and gene fusion. In some cases, an engineered or recombinant polynucleotide (e.g., a genomic DNA sequence) can be modified or altered by a gene editing moiety.

Unless specifically stated or obvious from context, the term “nucleotide” as used herein, generally refers to a base-sugar-phosphate combination. A nucleotide can comprise a synthetic nucleotide. A nucleotide can comprise a synthetic nucleotide analog. Nucleotides can be monomeric units of a nucleic acid sequence (e.g. deoxyribonucleic acid (DNA) and ribonucleic acid (RNA)). The term nucleotide can include ribonucleoside triphosphates adenosine triphosphate (ATP), uridine triphosphate (UTP), cytosine triphosphate (CTP), guanosine triphosphate (GTP) and deoxyribonucleoside triphosphates such as dATP, dCTP, dITP, dUTP, dGTP, dTTP, or derivatives thereof. Such derivatives can include, for example, [αS]dATP, 7-deaza-dGTP and 7-deaza-dATP, and nucleotide derivatives that confer nuclease resistance on the nucleic acid molecule containing them. The term nucleotide as used herein can refer to dideoxyribonucleoside triphosphates (ddNTPs) and their derivatives. Illustrative examples of dideoxyribonucleoside triphosphates can include, but are not limited to, ddATP, ddCTP, ddGTP, ddITP, and ddTTP. A nucleotide may be unlabeled or detectably labeled by well-known techniques. Labeling can also be carried out with quantum dots. Detectable labels can include, for example, radioactive isotopes, fluorescent labels, chemiluminescent labels, bioluminescent labels and enzyme labels. Fluorescent labels of nucleotides may include but are not limited fluorescein, 5-carboxyfluorescein (FAM), 2′7′-dimethoxy-4′5-dichloro-6-carboxyfluorescein (JOE), rhodamine, 6-carboxyrhodamine (R6G), N,N,N′,N′-tetramethyl-6-carboxyrhodamine (TAMRA), 6-carboxy-X-rhodamine (ROX), 4-(4′dimethylaminophenylazo) benzoic acid (DABCYL), Cascade Blue, Oregon Green, Texas Red, Cyanine and 5-(2′-aminoethyl) aminonaphthalene-1-sulfonic acid (EDANS). Specific examples of fluorescently labeled nucleotides can include [R6G]dUTP, [TAMRA]dUTP, [R110]dCTP, [R6G]dCTP, [TAMRA]dCTP, [JOE] ddATP, [R6G] ddATP, [FAM] ddCTP, [R110] ddCTP, [TAMRA] ddGTP, [ROX] ddTTP, [dR6G] ddATP, [dR110] ddCTP, [dTAMRA] ddGTP, and [dROX]ddTTP available from Perkin Elmer, Foster City, Calif. FluoroLink Deoxy Nucleotides, FluoroLink Cy3-dCTP, FluoroLink Cy5-dCTP, FluoroLink Fluor X-dCTP, FluoroLink Cy3-dUTP, and FluoroLink Cy5-dUTP available from Amersham, Arlington Heights, Ill.; Fluorescein-15-dATP, Fluorescein-12-dUTP, Tetramethyl-rodamine-6-dUTP, IR770-9-dATP, Fluorescein-12-ddUTP, Fluorescein-12-UTP, and Fluorescein-15-2′-dATP available from Boehringer Mannheim, Indianapolis, Ind.; and Chromosome Labeled Nucleotides, BODIPY-FL-14-UTP, BODIPY-FL-4-UTP, BODIPY-TMR-14-UTP, BODIPY-TMR-14-dUTP, BODIPY-TR-14-UTP, BODIPY-TR-14-dUTP, Cascade Blue-7-UTP, Cascade Blue-7-dUTP, fluorescein-12-UTP, fluorescein-12-dUTP, Oregon Green 488-5-dUTP, Rhodamine Green-5-UTP, Rhodamine Green-5-dUTP, tetramethylrhodamine-6-UTP, tetramethylrhodamine-6-dUTP, Texas Red-5-UTP, Texas Red-5-dUTP, and Texas Red-12-dUTP available from Molecular Probes, Eugene, Oreg. Nucleotides can also be labeled or marked by chemical modification. A chemically modified single nucleotide can be biotin-dNTP. Some non-limiting examples of biotinylated dNTPs can include, biotin-dATP (e.g., bio-N6-ddATP, biotin-14-dATP), biotin-dCTP (e.g., biotin-11-dCTP, biotin-14-dCTP), and biotin-dUTP (e.g. biotin-11-dUTP, biotin-16-dUTP, biotin-20-dUTP).

The term “cell” generally refers to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archaeal cell, a cell of a single-cell eukaryotic organism, a protozoa cell, a cell from a plant (e.g. cells from plant crops, fruits, vegetables, grains, soy bean, corn, maize, wheat, seeds, tomatoes, rice, cassava, sugarcane, pumpkin, hay, potatoes, cotton, cannabis, tobacco, flowering plants, conifers, gymnosperms, ferns, clubmosses, hornworts, liverworts, mosses), an algal cell, (e.g., Botryococcus braunii, Chlamydomonas reinhardtii, Nannochloropsis gaditana, Chlorella pyrenoidosa, Sargassum patens, C. Agardh, and the like), seaweeds (e.g. kelp), a fungal cell (e.g., a yeast cell, a cell from a mushroom), an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), and etcetera. Sometimes a cell is not originating from a natural organism (e.g., a cell can be a synthetically made, sometimes termed an artificial cell).

The term “reprogramming,” “dedifferentiation,” “increasing cell potency,” or “increasing developmental potency,” as used interchangeable herein, generally refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

The term “differentiation” generally refers to a process by which an unspecialized (“uncommitted”) or less specialized cell acquires the features of a specialized cell such as, e.g., an immune cell. A differentiated or differentiation-induced cell is one that has taken on a more specialized (“committed”) position within the lineage of a cell. The term “committed” generally refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type.

The term “pluripotent” generally refers to the ability of a cell to form all lineages of the body or soma (i.e., the embryo proper). For example, embryonic stem cells are a type of pluripotent stem cells that are able to form cells from each of the three germs layers, the ectoderm, the mesoderm, and the endoderm. Pluripotency can be a continuum of developmental potencies ranging from the incompletely or partially pluripotent cell (e.g., an epiblast stem cell), which is unable to give rise to a complete organism to the more primitive, more pluripotent cell, which is able to give rise to a complete organism (e.g., an embryonic stem cell).

The term “induced pluripotent stem cells” (iPSCs) generally refers to stem cells that are derived from differentiated cells (e.g., differentiated adult, neonatal, or fetal cells) that have been induced or changed (i.e., reprogrammed) into cells capable of differentiating into tissues of all three germ or dermal layers: mesoderm, endoderm, and ectoderm. The iPSCs produced do not refer to cells as they are found in nature. In some cases, iPSCs can be engineered to differentiation directly into committed cells (e.g., natural killer (NK) cells. In some cases, iPSCs can be engineered to differentiate first into tissue-specific stem cells (e.g., hematopoietic stem cells (HSCs) or hematopoietic progenitor cells), which can be further induced to differentiate into committed cells (e.g., NK cells).

The term “embryonic stem cell” (ESCs) generally refers to cells derived from the naturally occurring pluripotent stem cells of the inner cell mass of the embryonic blastocyst. Embryonic stem cells are pluripotent and give rise during development to all derivatives of the three primary germ layers: ectoderm, endoderm and mesoderm. In some cases, ESCs can be engineered to differentiate directly into committed cells (e.g., NK cells). In some cases, ESCs can be engineered to differentiate first into tissue-specific stem cells (e.g., HSCs), which can be further induced to differentiate into committed cells (e.g., NK cells).

The term “isolated stem cells” generally refers to any type of stem cells disclosed herein (e.g., ESCs, HSCs, mesenchymal stem cells (MSCs), etc.) that are isolated from a multicellular organism. For example, HSCs can be isolated from a mammal's body, such as a human body. In another example, an embryonic stem cells can be isolated from an embryo.

The term “isolated” generally refers to a cell or a population of cells, which has been separated from its original environment. For example, a new environment of the isolated cells is substantially free of at least one component as found in the environment in which the “un-isolated” reference cells exist. An isolated cell can be a cell that is removed from some or all components as it is found in its natural environment, for example, isolated from a tissue or biopsy sample. The term also includes a cell that is removed from at least one, some or all components as the cell is found in non-naturally occurring environments, for example, isolated form a cell culture or cell suspension. Therefore, an isolated cell is partly or completely separated from at least one component, including other substances, cells or cell populations, as it is found in nature or as it is grown, stored or subsisted in non-naturally occurring environments.

The term “hematopoietic stem and progenitor cells,” “hematopoietic stem cells,”, “hematopoietic progenitor cells,” or “hematopoietic precursor cells,” as used interchangeably herein, generally refers to cells which are committed to a hematopoietic lineage but are capable of further hematopoietic differentiation (e.g., into NK cells) and include, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). In some cases, HSCs can be CD34+ hematopoietic cells capable of giving rise to both mature myeloid and lymphoid cell types including T cells, NK cells and B cells.

The term “immune cell” generally refers to a differentiated hematopoietic cell. Non-limiting examples of an immune cell can include an NK cell, a T cell, a monocyte, an innate lymphocyte, a tumor-infiltrating lymphocyte, a macrophage, a granulocyte, etc.

### Overview

Biological programming, such as cellular programming, allows for the engineering of a cell to generate a desired outcome. Outcomes of cellular programming can include inducing or prevent a wide array of common and/or new cellular functions; outcomes can also include enhancing or repressing an already-occurring cellular function. Cellular programming can be accomplished through the use of a genetic circuit. Cellular programming can be accomplished through the manipulation of biomolecules (e.g., DNA). For example, CRISPR or CRISPR/Cas systems have been adopted for genome editing across many species due to its versatility and facile programmability. Cellular programming can affect endogenous or exogenous genes. Cellular programming can be implemented to function in a time-dependent manner or a time-independent manner.

Genetic circuits used in cellular programming can be used to control a cascade of a plurality of desired expression and/or activity profiles of a plurality of genes in the cell. To allow for better control of specific cellular outcomes, genetic circuits can be multiplexed to create positive feedback and/or negative feedback systems.

Although CRISPR/Cas systems are widely used for gene editing, Cas is essentially a single-turnover nuclease as it remains bound to the double-strand break it generates, and many regions of the genome are refractory to genome editing. Increased understanding of CRISPR/Cas-based genome editing has encouraged the development of cascading regulatory systems to further harness this technology for use in engineered cellular development. By implementing a series of activatable gRNA, genome editing can be regulated from target site to target site in more of a temporal manner, sequential genome edits can be executed to function like a domino effect, and cells can be barcoded. However, this simple barcoding, often using exogenous fluorophores, doesn't allow for the multiplexed regulation of endogenous genes to effect cell differentiation.

Thus, there remains an unmet need for an activatable, multiplexed CRISPR/Cas system and use of the same to edit a target polynucleotide (e.g., a genome of a cell, in particular a eukaryotic cell), using cascades of gRNAs to form genetic circuits which include feedback loops in order to single-handedly affect gene regulation and, in turn, cell-fate determination. Given its improved multiplexing capabilities through the use of internal positive and/or negative feedback loops, the preprogrammed, activatable, and self-regulating gRNA cascade CRISPR/Cas system finds use, e.g., in gene therapy, genetic circuitry, and/or complex cell-fate determination and/or control.

The present disclosure provides systems and methods for engineering a CRISPR/Cas9 system, which includes a Cas endonuclease and an array of cognate single guide RNAs (sgRNA or gRNA) that harbor inactivation sequences in a non-essential region and are activatable, to allow for modulation and modification of that system by the use of positive and negative feedback loops. The present disclosure also provides for an engineered cell that can contain any of the above-mentioned systems or that can be capable of performing any of the above-mentioned methods.

### Systems and Methods for Engineering Genetic Circuits, Cells Comprising Thereof, and Methods of Use Thereof

Various aspects of the present disclosure provide systems for inducing a desired expression and/or activity level (or profile thereof) of one or more target genes in a cell. Various aspects of the present disclosure provide methods for inducing a desired expression and/or activity level (or profile thereof) of one or more target genes in a cell.

In an aspect, the present disclosure provides for a system that induces a desired expression and/or activity profile of a target gene in a cell. The system can comprise a heterologous genetic circuit comprising a plurality of gate units. The plurality of gate units can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more gate unit(s). The plurality of gate units can comprise at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 gate unit(s). The plurality of gate units can be different (e.g., comprising different polynucleotide sequences).

A heterologous genetic circuit as disclosed herein can operate with a plurality of gate units in series (e.g., the plurality of gate units are connected sequentially in an end-to-end manner forming a single path), in parallel (e.g., the plurality of gate units are connected across one another, forming, for example, two or more parallel paths), or a combination thereof.

A plurality of gate units as disclosed herein can operate (e.g., as predetermined by the design of the heterologous genetic circuit) in concert to induce an outcome in a cell. The outcome in the cell can comprise cell function (e.g., movement, reproduction; response to external stimuli, nutritional output, excretion, respiration, growth) and/or cell state (e.g., cell fate, differentiation, quiescence, programmed cell death). Such outcomes can be ascertained in vitro, ex vivo, and/or in vivo. For example, an outcome as disclosed herein can be ascertained in vitro by (i) measuring expression level of a gene of interest by polymerase chain reaction (PCR) or Western blotting, (ii) staining via small molecules or antibodies, (iii) cell sorting based on cell size, morphology and/or surface protein expression, (iv) using assays (e.g. cell proliferation assays, metabolic activity assays, cell killing assays) to measure phenotypic differentiation and cellular function, (v) microscopy, and/or (iv) screening for molecular and/or genetic differences using e.g., metabolomics, genomics, proteomics, lipidomics, epigenomics, and/or transcriptomics.

The outcome in the cell can comprise regulation of a target gene. The regulation of the target gene can comprise a plurality of distinct modulations of the target gene. The plurality of gate units can each induce one of the plurality of distinct modulations of the target gene, such that a collection of the distinct modulation in concert yields a final expression and/or activity profile of the target gene. At least two distinct modulations of the plurality of distinct modulations can both increase an expression and/or activity level of the target gene. At least two distinct modulations of the plurality of distinct modulations can both decrease an expression and/or activity level of the target gene. Alternatively, a first distinct modulation of the plurality of distinct modulations can increase an expression and/or activity level of the target gene, while a second distinct modulation of the plurality of distinct modulations can decrease the expression and/or activity level of the target gene. In such case, the first distinct modulation can occur prior to the second distinct modulation, or vice versa. Alternatively, a distinct modulation (e.g., a first and/or second modulation) of the plurality of distinct modulations can maintain an expression and/or activity level of the target gene at the level of expression and/or activity level prior to the modulation.

In some cases, each distinct modulation of the plurality of distinct modulations of the target gene, as disclosed herein, can be necessary but individually insufficient to effect the desired expression and/or activity profile of the target gene. Thus, the outcome in the cell (e.g., enhanced cell function, induced cell state, etc.) induced by the plurality of distinct modulations of the target gene may not be possible in absence of any one of the plurality of distinct modulations of the target gene. Alternatively, a degree or measure of the outcome in the cell induced by the plurality of distinct modulations of the target gene can be greater than a degree or measure of the outcome in a control cell that is induced by none, one or more, but not all of the plurality of distinct modulations of the target gene, and/or by all of the plurality of distinct modulation of the target genes occurring through a different sequential order of events.

A second gate unit can be activated by a first gate unit (e.g. directly or indirectly). For example, the second gate unit can be directly activated by the first gate unit. Alternatively, the second gate unit can be activated by one or more additional gate units that are activated by the first gate unit (e.g., directly or indirectly). The one or more additional gate units can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50 or more gate unit(s). The one or more additional gate units at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 gate unit(s). Yet in another alternative, the second gate unit can be activated via another moiety responsible for activating the first gate unit (e.g., an activating moiety, a different gate unit, etc.).

The second gate unit can be activatable to induce inactivation of the first gate unit that has been activated. The terms “inactivation” or “disruption” may be used interchangeably herein. Inactivation and as disclosed herein can be induced by generating a modification (e.g., a cleavage such as a single-strand or double-strand break, and indel, etc.) to at least a portion of the first gate unit (e.g. a gate moiety and/or a gene regulating moiety of the first gate unit) that is responsible for inducing the first distinct modulation of the target gene.

Inactivation by a gate moiety and/or a gene regulating moiety of the first gate unit as disclosed herein can be achieved through a endonuclease-based system (e.g., a CRISPR/Cas system). Alternatively or in addition to, inactivation can be achieved through the use of a transcriptional modulator system (e.g. a transcriptional repressor). An endonuclease-transcriptional modulator system (e.g., a Cas-repressor) can be used to achieve polynucleotide cleavage (e.g. for inactivating the gate moiety and/or the gene regulating moiety). Polynucleotide cleavage can create a nucleic acid modification such as a single-strand break, a double-strand break, an insertion, a deletion, or an insertion-deletion (indel). Alternatively or in addition to, the endonuclease-transcriptional modulator system (e.g., a Cas-repressor) can be used to modulate target gene expression.

Alternatively, the second gate unit can be activatable to amplify or enhance activation of the first gate unit that has been activated. Amplification or enhancement of the first gate unit can be induced by generating a modification (e.g., a cleavage such as a single-strand or double-strand break, and indel, etc.) to at least a portion of the first gate unit (e.g. a gate moiety and/or a gene regulating moiety of the first gate unit) that is responsible for inducing the first distinct modulation of the target gene.

In some cases, a guide nucleic acid molecules (gNA) (e.g., a functional gNA) that is expressed by the second gate unit, upon activation, can create a modification to at least a portion of the first gate unit. For example, the activated gNA of the second gate unit can generate the modification to a polynucleotide sequence of the first gate unit that encodes a gNA (e.g., an activatable gNA) or a promoter sequence of the first gate unit that is operatively coupled to such gNA of the same first gate unit. Such modification can render the gNA of the first gate unit inoperable when expressed (e.g., reduced or inhibited specific binding to the target gene). Alternatively, the modification can reduce (e.g., inhibit) expression of the gNA of the first gate unit.

In some cases, modification of a polynucleotide sequence (e.g., as a component of a gate unit, such as a gate moiety) or a target gene can be caused by a single-stranded break wherein there is a discontinuity in one nucleotide strand. Inactivation of a polynucleotide sequence or a target gene can be caused by at least 1, at least 2, at least 3, at least 4, at least 5, at least 6, at least 7, at least 8, at least 9, at least 10, or more single-stranded breaks. In some cases, inactivation of a gene can be caused by at most 10, at most 9, at most 8, at most 7, at most 6, at most 5, at most 4, at most 3, at most 2, or at most 1 single-stranded breaks.

In some cases, a gNA is at least about 10 nucleotides, at least about 11 nucleotides, at least about 12 nucleotides, at least about 13 nucleotides, at least about 14 nucleotides, at least about 15 nucleotides, at least about 16 nucleotides, at least about 17 nucleotides, at least about 18 nucleotides, at least about 19 nucleotides, at least about 20 nucleotides, at least about 21 nucleotides, at least about 22 nucleotides, at least about 23 nucleotides, at least about 24 nucleotides, at least about 25 nucleotides, at least about 26 nucleotides, at least about 27 nucleotides, at least about 28 nucleotides, at least about 29 nucleotides, at least about 30 nucleotides or more in length.

In some cases, the system and methods of the present disclosure can utilize at least two different endonucleases. A first endonuclease (e.g., a Cas protein that is not coupled to a transcriptional modulator) can be used in conjunction with a guide nucleic acid to induce a cleavage of a target polynucleotide sequence of a gate moiety or a gene regulating moiety plasmids, to activate and/or deactivate the gate moiety or the gene regulating moiety, respectively. In addition, a second endonuclease (e.g., a Cas protein that is coupled to a transcriptional modulator) can be used in conjunction with another guide nucleic acid to bind to a target gene (e.g., a target endogenous gene) of a cell, to modulate expression and/or activity of the target gene without generating a cleavage in the target gene.

In some cases, the systems and methods of the present disclosure can utilize a single endonuclease system (e.g., a Cas-repressor) to achieve both (i) polynucleotide cleavage (e.g. for activating/inactivating the gate moiety and/or the gene regulating moiety) and (ii) modulation of target gene expression. When using a single endonuclease-transcriptional modulator system, unique guide nucleic acid molecules (gNAs) of differing spacer sequence lengths can be used to determine whether the single endonuclease-transcriptional modulator system may (i) hybridize to the polynucleotide sequence to induce Cas-mediated nuclease activity of the polynucleotide sequence, or (ii) can hybridize to a target gene (e.g., genomic DNA) to modulate expression and/or activity level of the target gene via action of the transcriptional activator without mediating Cas nuclease activity, as desired by the individual heterologous genetic circuit. For example, use of gNAs of differing spacer sequence lengths that bind to different targets can allow for a second gate unit as provided herein to induce inactivation of a first gate unit that has been activated and/or induce a distinct modulation of a second target gene.

As abovementioned, the length the spacer sequence of the gNA can affect the ability of the gNA to mediate Cas nuclease activity. In some cases, gNAs with spacer sequences of differing lengths can be used in the same heterologous genetic circuit to affect different types of cleavage, activation, inactivation, and/or modulation of one or more target nucleic acids. In some cases, a gNA spacer sequence that is shorter than a threshold length (e.g., about 16 nucleotides) can preclude nuclease activity of a Cas-transcriptional modulator, while still mediating DNA binding for transcriptional modulation of a target gene. In some cases, a gNA spacer sequence that is shorter than at least about 25 nucleotides, at least about 20 nucleotides, at least about 19 nucleotides, at least about 18 nucleotides, at least about 17 nucleotides, at least about 16 nucleotides, at least about 15 nucleotides, at least about 15 nucleotides, at least about 14 nucleotides, at least about 13 nucleotides, at least about 12 nucleotides, at least about 11 nucleotides, or at least about 10 nucleotides can preclude nuclease activity of a Cas protein while still mediating DNA binding.

For example, a gNA comprising a 20-nucleotide spacer sequence (e.g., a gNA encoded by a gate moiety for targeting a gene regulating moiety plasmid) can be sufficient to facilitate nuclease activity of an endonuclease (e.g. a Cas or a Cas-transcriptional modulator fusion protein) at a target polynucleotide sequence. Alternatively or in addition to, a gNA comprising a 14-nucleotide spacer sequence (e.g., a gNA encoded by a gene regulating moiety) can hybridize to DNA but may not be long enough to mediate nuclease activity—it can only facilitate endonuclease binding to the cognate DNA sequence. Accordingly, the shorter gNA can selectively allow for transcriptional modulation of a target gene though the use of a endonuclease-transcriptional modulator system (e.g. a Cas-activator system, a Cas-repressor system), without cleavage of the target gene.

In some cases, modification of a polynucleotide sequence (e.g., as a component of a gate unit, such as a gate moiety) or a target gene can be caused by a double-stranded break wherein there is a discontinuity in both nucleotide strands. In some cases, inactivation of a gene can be caused by at least 1, at least 2, at least 3, at least 4, at least 5, at least 6, at least 7, at least 8, at least 9, at least 10, or more double-stranded breaks. In some cases, inactivation of polynucleotide sequence or a target gene can be caused by at most 10, at most 9, at most 8, at most 7, at most 6, at most 5, at most 4, at most 3, at most 2, or at most 1 double-stranded breaks.

In some cases, modification of a polynucleotide sequence (e.g., as a component of a gate unit, such as a gate moiety) or a target gene can be caused by an indel, also known as an insertion-deletion mutation. An indel mutation can comprise a frameshift or non-frameshift mutation. An indel mutation can comprise a point mutation, also called a base substitution, wherein only one base or base pair is modified. An indel mutation can comprise at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, at least about 60, at least about 70, at least about 80, at least about 90, at least about 100, at least about 200, at least about 300, at least about 400, at least about 500, at least about 600, at least about 700, at least about 800, at least about 900, at least about 1000, at least about 2000, or more bases or base pairs in length. An indel mutation can comprise at most about 2000, at most about 1000, at most about 900, at most about 800, at most about 700, at most about 600, at most about 500, at most about 400, at most about 300, at most about 200, at most about 100, at most about 90, at most about 80, at most about 70, at most about 60, at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 bases or base pairs in length.

In some cases, modification of a polynucleotide sequence (e.g., as a component of a gate unit, such as a gate moiety) or a target gene can be achieved without cleavage of the polynucleotide sequence or the target gene. For example, a gene regulating moiety (e.g., a nucleic acid molecule and/or an endonuclease, such as a complex comprising a CRISPR/Cas protein and a guide nucleic acid molecule) can specifically bind to the polynucleotide sequence or the target gene, such that expression and/or activity of the polynucleotide sequence or the target gene is modified. The gene regulating moiety can comprise a transcriptional repressor or a transcriptional activator, as provided herein.

In some cases, modification of a polynucleotide sequence (e.g., as a component of a gate unit, such as a gate moiety) or a target gene can inactivate a gene. For example, modification of a polynucleotide sequence or a target gene can stop expression and/or activity level of the polynucleotide sequence or the target gene. Alternatively, modification of a polynucleotide sequence or a target gene can decrease the expression and/or activity level of the polynucleotide sequence of the target gene. In some cases, modification of a polynucleotide sequence or a target gene can increase the expression and/or activity level of the polynucleotide sequence or the target gene. Alternatively, modification of a polynucleotide sequence or a target gene can maintain the expression and/or activity level of the polynucleotide sequence or the target gene.

In some cases, modification of a polynucleotide sequence (e.g., as a component of a gate unit, such as a gate moiety) or a target gene can comprise decreasing the expression and/or activity level of the polynucleotide sequence or the target gene, respectively, by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, or more. Modification of a polynucleotide sequence or a target gene can comprise decreasing the expression and/or activity level of the polynucleotide sequence or the target gene, respectively, by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a polynucleotide sequence (e.g., as a component of a gate unit, such as a gate moiety) or a target gene can comprise increasing the expression and/or activity level of the polynucleotide sequence or the target gene, respectively, by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, at least about 1,000,000% or more. Modification of a polynucleotide sequence or a target gene can comprise increasing the expression and/or activity level of the polynucleotide sequence or the target gene, respectively, by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

In some cases, modification of a polynucleotide sequence (e.g., as a component of a gate unit, such as a gate moiety) or a target gene can comprise decreasing the expression and/or activity level of the polynucleotide sequence or the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. Modification of a polynucleotide sequence or a target gene can comprise decreasing the expression and/or activity level of the polynucleotide sequence or the target gene, respectively, by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

In some cases, modification of a polynucleotide sequence (e.g., as a component of a gate unit, such as a gate moiety) or a target gene can comprise increasing the expression and/or activity level of the polynucleotide sequence or the target gene, respectively, by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. Modification of a polynucleotide sequence or a target gene can comprise increasing the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

In some cases, (i) a change (e.g., enhancement or reduction) in the expression and/or activity level of the target gene upon the plurality of distinct modulations of the target gene in the sequential manner can be greater than (ii) any change in expression and/or activity level of the target gene upon only one or none of the plurality of distinct modulations, by at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 11%, at least or up to about 12%, at least or up to about 13%, at least or up to about 14%, at least or up to about 15%, at least or up to about 16%, at least or up to about 17%, at least or up to about 18%, at least or up to about 19%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, at least or up to about 100%, at least or up to about 125%, at least or up to about 150%, at least or up to about 175%, at least or up to about 200%, at least or up to about 225%, at least or up to about 250%, at least or up to about 275%, or at least or up to about 300%.

In some cases, (i) a change (e.g., enhancement or reduction) in the expression and/or activity level of the target gene upon the plurality of distinct modulations of the target gene in the sequential manner can last for a longer duration than (ii) any change in expression and/or activity level of the target gene upon only one or none of the plurality of distinct modulations, by at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 11%, at least or up to about 12%, at least or up to about 13%, at least or up to about 14%, at least or up to about 15%, at least or up to about 16%, at least or up to about 17%, at least or up to about 18%, at least or up to about 19%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least or up to about 95%, at least or up to about 100%, at least or up to about 125%, at least or up to about 150%, at least or up to about 175%, at least or up to about 200%, at least or up to about 225%, at least or up to about 250%, at least or up to about 275%, or at least or up to about 300%.

In some cases, (i) the expression and/or activity level of the target gene upon the plurality of distinct modulations of the target gene in the sequential manner (e.g., including the sequential disruption) can be less than (ii) expression and/or activity level of the target gene upon only one the first distinct modulation but not the second distinct modulation, by at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 11%, at least or up to about 12%, at least or up to about 13%, at least or up to about 14%, at least or up to about 15%, at least or up to about 16%, at least or up to about 17%, at least or up to about 18%, at least or up to about 19%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least up to or about 91%, at least up to or about 92%, at least up to or about 93%, at least up to or about 94%, at least up to or about 95%, at least up to or about 96%, at least up to or about 97%, at least up to or about 98%, at least up to or about 99%, or substantially about 100%.

In some cases, (i) the expression and/or activity level of the target gene upon the plurality of distinct modulations of the target gene in the sequential manner (e.g., including the sequential disruption) can last shorter than (ii) expression and/or activity level of the target gene upon only one the first distinct modulation but not the second distinct modulation, by at least or up to about 1%, at least or up to about 2%, at least or up to about 3%, at least or up to about 4%, at least or up to about 5%, at least or up to about 6%, at least or up to about 7%, at least or up to about 8%, at least or up to about 9%, at least or up to about 10%, at least or up to about 11%, at least or up to about 12%, at least or up to about 13%, at least or up to about 14%, at least or up to about 15%, at least or up to about 16%, at least or up to about 17%, at least or up to about 18%, at least or up to about 19%, at least or up to about 20%, at least or up to about 25%, at least or up to about 30%, at least or up to about 35%, at least or up to about 40%, at least or up to about 45%, at least or up to about 50%, at least or up to about 55%, at least or up to about 60%, at least or up to about 65%, at least or up to about 70%, at least or up to about 75%, at least or up to about 80%, at least or up to about 85%, at least or up to about 90%, at least up to or about 91%, at least up to or about 92%, at least up to or about 93%, at least up to or about 94%, at least up to or about 95%, at least up to or about 96%, at least up to or about 97%, at least up to or about 98%, at least up to or about 99%, or substantially about 100%.

In some cases, the first gate unit and the second gate unit as provided here can be activated to induce a first distinct modulation and a subsequent second distinct modulation of a common gene (e.g., expression and/or activity profile of the common gene). The first gate unit and the second gate unit may be activated sequentially (e.g., at different time points), to effect the first and second distinct modulations of the common gene in a sequential manner. Alternatively, the first gate unit and the second gate unit may be activated subsequently simultaneously, and such gates may be preconfigured to still effect the first and second distinct modulations of the common gene in such sequential manner.

In some cases, activation of the plurality of gate units may be a result of a single activation (e.g., by a single activating moiety at a single time point) of the heterologous genetic circuit. The plurality of gate units can comprise one of the first gate unit and the second gate that are preconfigured to be activated sequentially upon activation of the heterologous genetic circuit by the single activation. In some cases, one of the first and second gate unit can be activated by the single activating moiety (e.g., a guide nucleic acid), while the other of the first and second gate unit can be activated by an additional activating moiety (e.g., a different guide nucleic acid) that is different from the activating moiety of the heterologous genetic circuit. The additional activating moiety can be a part of the heterologous genetic circuit that is generated (e.g., expressed) only upon activation of the heterologous genetic circuit. Alternatively or in addition to, the first and second gate unit can each be activated by different activating moieties that are not the same as the activating moiety of the heterologous genetic circuit. Such different activating moieties can be parts of the heterologous genetic circuit that are generated (e.g., expressed) only upon activation of the heterologous genetic circuit.

In some embodiments of any one of the systems disclosed herein, a gate unit can comprise a gate moiety (e.g., at least 1, 2, 3, 4, 5, or more different gate moieties) and/or a gene regulating moiety (e.g., at least 1, 2, 3, 4, 5, 6, 7, 8, 9, 10, or more different gene regulating moieties). A gate moiety as disclosed herein can comprise a guide nucleic acid molecule (gNA) (e.g., at least 1, 2, 3, 4, 5, or more gNAs). A gene regulating moiety as disclosed herein can comprise a gNA (e.g., at least 1, 2, 3, 4, 5, or more gNAs). The guide nucleic acid molecule as disclosed herein can comprise, but is not limited to, DNA, RNA, any analog of such, or any combination thereof. In some embodiments of any one of the systems disclosed herein, the gate moiety and/or the gene regulating moiety can be activatable to form a complex with an enzyme (e.g., an endonuclease and/or an exonuclease), and the complex can be configured to or capable of binding a target polynucleotide, e.g., to regulate expression and/or activity level of the target polynucleotide or another polynucleotide sequence operatively coupled to the target polynucleotide. For example, the complex can regulate expression and/or activity level of a gene comprising the target polynucleotide.

In some embodiments of any one of the systems disclosed herein, an initial (or the first) gate unit of the heterologous genetic circuit as disclosed herein may be activated (e.g., directly activated) by an activating moiety. The activating moiety can directly bind at least the portion of the initial gate unit to activate the initial gate unit, e.g., thereby to sequentially activate the heterologous genetic circuit. Alternatively, the activating moiety (e.g., electromagnetic energy) may activate the initial gate unit without directly binding the at least the portion of the initial gate unit. In some cases, the initial gate unit can comprise at least one gate moiety and at least one gene regulating moiety. In some cases, the initial gate unit can comprise at least one gate moiety but may not and need not comprise a gene regulating moiety. In some cases, the initial gate unit can comprise at least one gene regulating moiety but may not and need not comprise a gate moiety (e.g., the activating moiety may be configured to activate the initiate gate unit and at least one additional gate unit).

In some embodiments of any one of the systems disclosed herein, the gNA of the gate moiety and/or the gene regulating moiety (e.g., a gNA encoded by the gate moiety and/or the gene regulating moiety) can be an activatable gNA. The activatable gNA can be one of, but not limited to, any of the following: ribonucleotides (e.g., gRNA), deoxyribonucleotides, any analog of such, or any combination thereof. In some embodiments, the activatable gNA molecule can be a self-cleaving gNA (e.g., the gRNA contains a cis ribozyme). For example, when the activatable gNA is expressed in a cell, the activatable gNA may be self-cleavable to become non-functional (e.g., not configured to bind a target gene), unless a gene encoding the activatable gNA is modified prior to the expression of the activatable gNA. In some embodiments, the activatable gNA molecule comprises a non-canonical transcription termination sequence (e.g., a polyX sequence, such as a polyU sequence or a polyT sequence), such that a functional gNA molecule is not expressed until a gene encoding the activatable gNA having the non-canonical transcription termination sequence can be modified (e.g., to remove some or all of the transcription termination sequence). Thus, in absence of the modification of the transcription termination sequence, a non-functional variant (e.g., a non-functional fragment) of the gNA may be expressed. In some embodiments, the gNA can be synthetic. In some embodiments, the gNA can have a fluorescent label attached.

In some cases, a size of the polyT sequence is greater than or equal to a threshold length, wherein the threshold length is sufficient to reduce expression of the guide nucleic acid molecule from the polynucleotide sequence. Accordingly, a plasmid (e.g., a gate moiety or a gene regulating moiety) can encode an inactivated gNA comprising the polyT sequence that is greater than or equal to the threshold length, and editing of such plasmid to reduce the length of the polyT to below the threshold length can permit expression of the gNA in its entirety without early termination, thereby activating the gNA. In some cases, the polyT sequence comprises at least 5 T. In some cases, the polyT sequence comprises at least 7 T. In some cases, the polyT sequence comprises at least 8 T. In some cases, the polyT sequence comprises at least 10 T. In some cases, the polyT sequence comprises between 5 T and 15 T. In some cases, the polyT sequence comprises one or more additional nucleotides that are not T.

In some cases, a gene regulating moiety (e.g., a guide nucleic acid and/or an endonuclease) can be configured to bind to a target polynucleotide sequence operatively coupled to a target gene in a cell. The target gene can comprise an encoding polynucleotide sequence that encodes a target nucleic acid molecule or a target protein. The target polynucleotide sequence can be a part of the encoding polynucleotide sequence. Alternatively, the target polynucleotide sequence may not be a part of the encoding polynucleotide sequence. For example, the target polynucleotide sequence can be upstream of the encoding polynucleotide sequence (e.g., part of a promoter of the encoding polynucleotide sequence, such as a transcription start site (TSS).

The plurality of distinct modulations of the target gene can be different (e.g., different degrees of change in the expression and/or activity level of the target gene. For example, a first modulation exerted by a first gene unit and second modulation exerted by a second gate unit can be different by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, or at least about 500%. The first modulation and the second modulation can be different by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.1%. Alternatively or in addition to, the distinct modulation of the target gene can be substantially the same (e.g., the same).

The plurality of distinct modulations can be individually sufficient to induce the desired change in expression and/or activity level of the target gene. Alternatively, the distinct modulations can be individually insufficient to induce the desired change in expression and/or activity level of the target gene.

One or more target genes as disclosed herein can comprise one or more endogenous genes (e.g., genomic DNA, mRNA, mitochondrial DNA, etc.), exogenous genes, transgenes, or a combination thereof.

One or more target genes as disclosed herein can comprise a cell differentiation regulatory factor, a molecular function regulatory factor, a binding factor, a fusogenic factor, a protein folding chaperone, a protein tag, a RNA folding chaperone, a cell signaling factor, an immune response factor, a sensory receptor, a cell structural factor, a protein binding factor, a cargo receptor, a catalytic factor, or a small molecule sensor.

One or more target genes as disclosed herein can comprise a cell differentiation regulatory factor that comprises a growth factor, a transcription factor, a myogenic regulatory factor, an immune cell regulatory factor, a neuronal regulatory factor, a stem cell differentiation factor, a chondrogenic regulatory factor, an osteogenic regulatory factor, a senescence factor, a stemness factor (e.g., de-differentiation factor), etc.

In some cases, the one or more target genes (e.g., one or more myogenic regulatory factors) can comprise Msgn1, Pou5f1, Pax7, Myog, Myf5, Myf6, Myof, Srf, Ccnd2, Fgf2, Sox2, Tbx6, Tbxt, Ctnnb1, Mycl, Bcl212, Bcl211, Bcl2a1, Myod1, Bcl2, Tdgf1, Pax3, Chrd, Cond3, Hgf, Cond1, Foxm1, Myc, Tgfb1, Mdm2, Mcl1, Igf1, Mef2c, Mrf4, MyHC, MCK, Six1, Six4, Hdac4, Notch1, Ezh2, p21, Myh1, Myh2, Myh4, and/or Smad4.

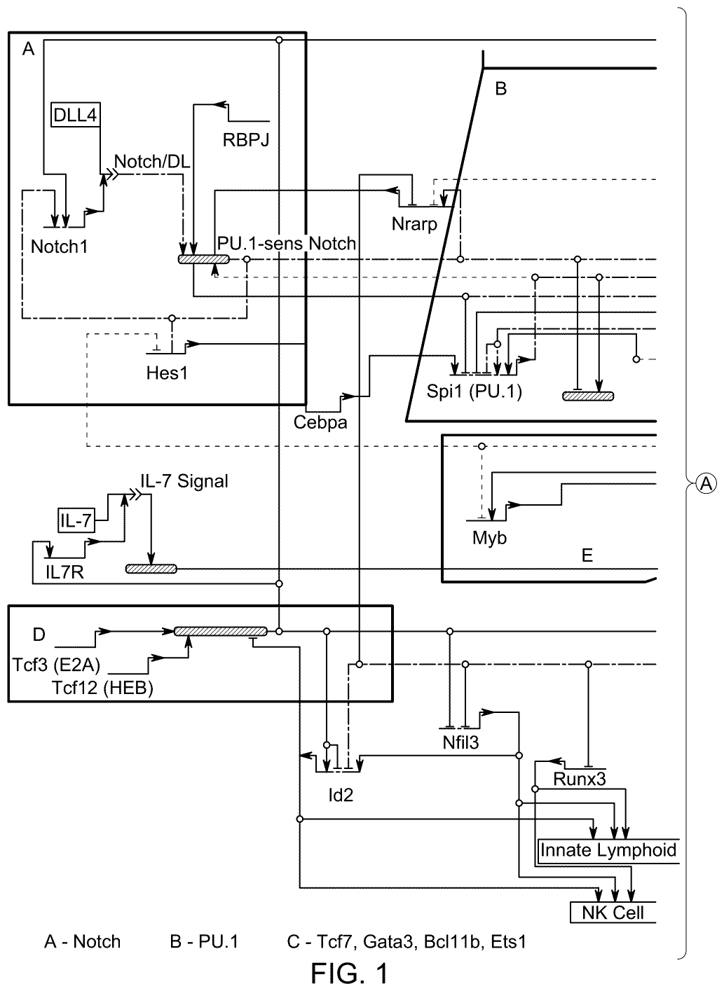

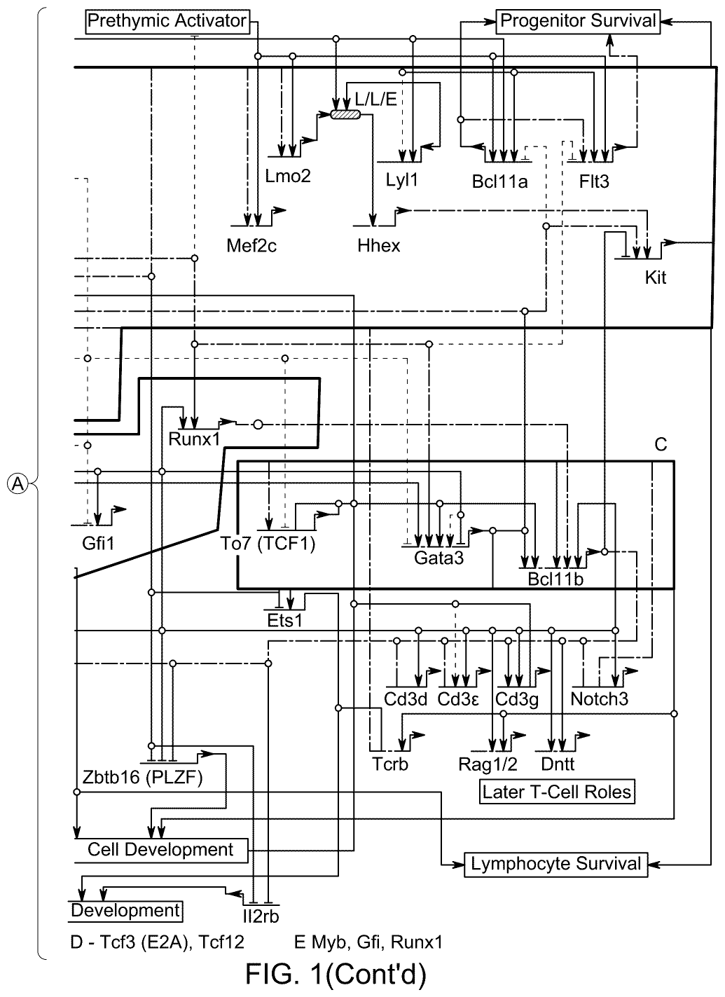

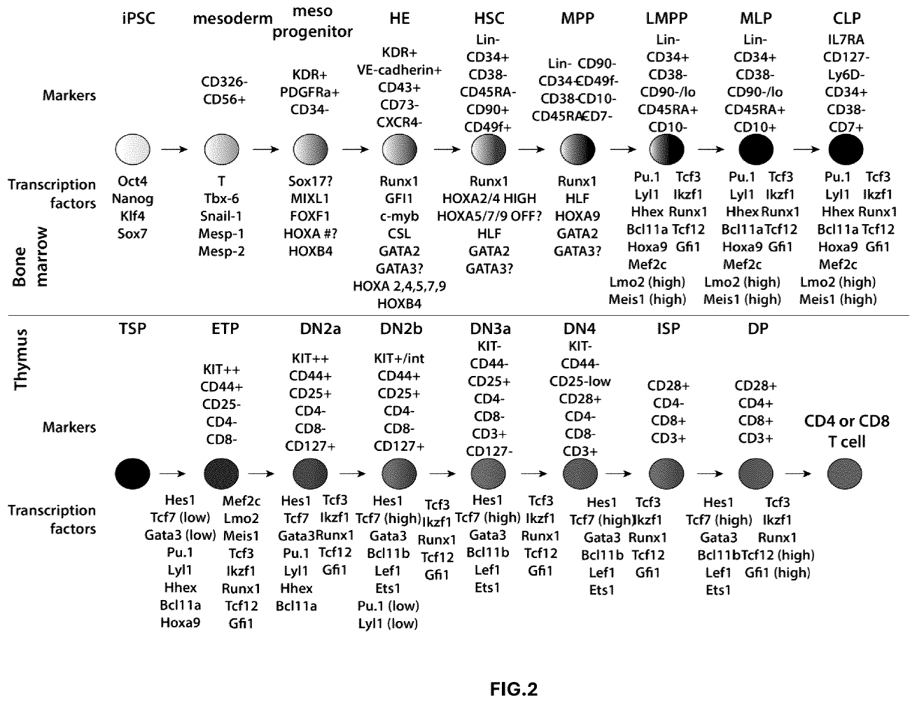

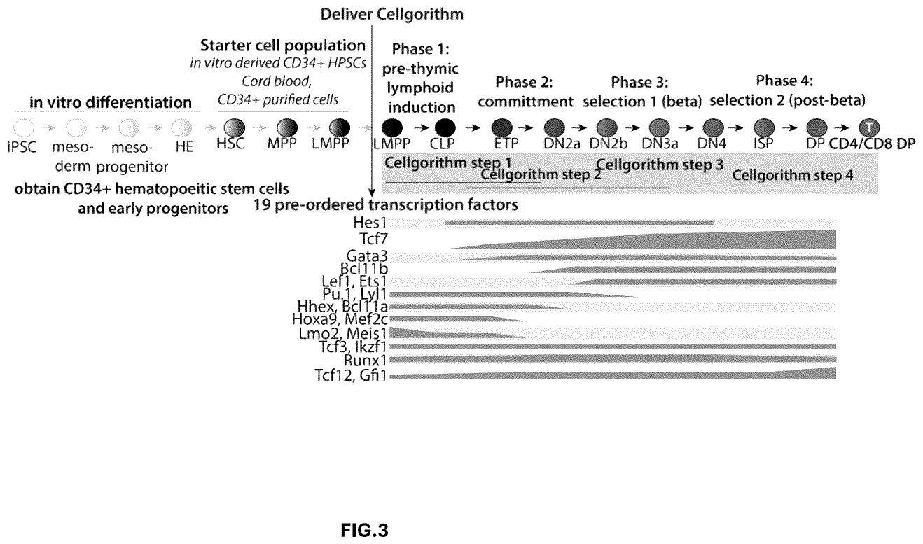

In some cases, the one or more target genes (e.g., one or more immune cell regulatory factors) can comprise Mycn, Ptcra, Bcl11b, Hhex, Notch1, Tcf3, Rag2, Dtx1, Runx1, Hoxa9, Rag1, Spi1, Ets1, Id2, Bcl11a, Id3, Tcf7, Ikzf1, Tcf12, Runx3, Lmo2, Lef1, Gfil, Lyl1, Aqp3, Meis1, Gata3, Hes1, St18, Nr4a1, C20orf100, Ikaros, Spib, Rorc, Tef1, Myc, Ahr, Foxo1, Notch1, Notch3, IL-2, IL4, IL7, IL 15 Ebf1, Pax5, Tal1, Myb, Erg, Hhex, E4pb4, Gfilb, pTa, Tbx21 Ets1, Ets2, or Mef2c (FIGS. 1-2). For example, one or more target genes for inducing T cell differentiation can be selected from the group consisting of Hes1, Gata3, Tcf1, Tcf3, Tcf7, Spi1, Lmo2, Ikzf1, Notch1, IL7, IL2, Runx1, and Bcl11b (FIG. 3). In another example, one or more target genes for inducing B cell differentiation can be selected from the group consisting of Ebf1, IL4, Tcf3, Foxo1, and Pax5. In another example, one or more target genes for inducing NK cell differentiation can be selected from the group consisting of E4bp4, Spi1, ZBTB16, Tbx21, IL7, IL 15, and Notch1.

In some cases, the one or more target genes (e.g., one or more stem cell differentiating factors) can comprise HoxB4, Notch1, Sox11, Sox17, Runx1, Gata2, Fli1, Erg, Wnt, Hnf1, Hnf3, Hnf4, Cdx2, and Lin28A.

In some cases, the one or more target genes (e.g., one or more neuronal regulatory factors) can comprise Ezh2, Sox1, Sox 3, Sox4, Sox9, Sox10, Sox11, Sox21, Hes1, Dlk1, Ascl1, Ngn1, Ngn2, Lhx6, Zeb1, Zeb2, Smad7, Myt11, Pax6, Wnt7a, Nptx1, Nab2, Lhx6, Olig2, Nfia, Nfib, Dlx5, Nurr1, Gpx1, Otx2, Pbx1, Foxa2, Isl1, Phox2a, Phox2b, Pitx3, Brn3a, and Brn4.

In some cases, the one or more target genes (e.g. one or more chondrogenic regulatory factors) can comprise Sox9, Gli3, Trps1, Nkx3.2, Runx2, Runx3, Smad1, Smad5, and Smad8.

In some cases, the one or more target genes (e.g. one or more osteogenic regulatory factors) can comprise Fos, Crebbp, Hnf4a, Irf1, Irf9, Foxo4, Meis1, Egr1, Pou2f1, Anf281, Spl, Esrl, Sox9, Znf6, Znf162, and Znf281.

In some cases, the one or more target genes (e.g., one or more senescence factors) can comprise p21, p53, Bax, Apaf1, Mdm2, E2f1, and Foxo3.

In some cases, the one or more target genes (e.g., one or more stemness factors) can comprise Hes1, Hes5, Cbf1, Soc2, Hmga2, Olig2, Id2, Id4, Hesr1, Hesr2, Gli1, Gli2, Gli3, SoxB, and Bmi1.

In some cases, the one or more target genes can comprise T-box transcription factors (TBX genes). TBX transcription factors are involved in development. T-box proteins have relatively large DNA-binding domains. Non-limiting examples of TBX transcription factors can include TBX1, TBX2, TBX3, TBX4, TBX5, TBX6, TBX10, TBX15, TBX18, TBX19, TBX20, TBX21, TBX22, and TBXT (Brachyury protein).

In some cases, the one or more target genes can comprise a basic helix-loop-helix transcription factor (bHLH gene). bHLH transcription factors are involved in the regulation of the cell cycle and many other developmental processes. bHLH proteins have a basic helix-loop-helix protein structure. Non-limiting examples of bHLH transcription factors can include AHR, AHRR, ARNT, ARNT2, ARNTL, ARNTL2, ASCL1, ASCL2, ASCL3, ASCL4, ATOH1, ATOH7, ATOH8, BHLHB2, BHLHB3, BHLHB4, BHLHB5, BHLHB8, CLOCK, EPASI, FERD3L, FIGLA, HAND1, HAND2, HES1, HES2, HES3, HES4, HES5, HES6, HES7, HEY1, HEY2, HIF1A, ID1, ID2, ID3, ID4, KIAA2018, LYL1, MASH1, MATH2, MAX, MESP1, MESP2, MIST1, MITF, MLX, MLXIP, MLXIPL, MNT, MSC, MSGN1, MXD1, MXD3, MXD4, MXI1, MYC, MYCL1, MYCL2, MYCN, MYF5, MYF6, MYOD1, MYOG, NCOAI, NCOA3, NEUROD1, NEUROD2, NEUROD4, NEUROD6, NEUROG1, NEUROG2, NEUROG3, NHLH1, NHLH2, NPAS1, NPAS2, NPAS3, NPAS4, OAF1, OLIG1, OLIG2, OLIG3, PTFIA, SCL, SCXB, SIM1, SIM2, SOHLH1, SOHLH2, SREBF1, SREBF2, TAL1, TAL2, TCF12, TCF15, TCF21, TCF3, TCF4, TCFL5, TFAP4, TFE3, TFEB, TFEC, TWIST1, TWIST2, USF1, and USF2.

In some cases, the one or more target genes can comprise a SRY-related box transcription factor (SOX gene). SOX transcription factors are involved in developmental regulation. Non-limiting examples of SOX transcription factors can include SOX1, SOX2, SOX3, SOX4, SOX5, SOX6, SOX7, SOX8, SOX9, SOX10, SOX11, SOX12, SOX13, SOX14, SOX15, SOX17, SOX18, SOX21, SOX30, and SRY.

In some cases, the one or more target genes can comprise SOX group A, comprising SRY. In some cases, the one or more target genes can comprise SOX group B1, comprising SOX1, SOX2 and/or SOX3. In some cases, the one or more target genes can comprise SOX group B2, comprising SOX14 and/or SOX21. In some cases, the one or more target genes can comprise SOX group C, comprising SOX4, SOX11 and/or SOX12. In some cases, the one or more target genes can comprise SOX group D, comprising SOX5, SOX6, and/or SOX13. In some cases, the one or more target genes can comprise SOX group E, comprising SOX8, SOX9, and/or SOX10. In some cases, the one or more target genes can comprise SOX group F, comprising SOX7, SOX 17, and/or SOX18. In some cases, the one or more target genes can comprise SOX group G, comprising SOX15. In some cases, the one or more target genes can comprise SOX group H, comprising SOX30.

In some cases, the one or more target genes can comprise a forkhead box (FOX). FOX are transcription factors that play a role in regulating the expression of genes involved in cell growth, proliferation, differentiation, and longevity. Some FOX genes can bind chromatin during cell differentiation processes. Non-limiting examples of FOX genes can include FOXA, FOXB, FOXC, FOXD, FOXE, FOXF, FOXG, FOXH, FOXI, FOXJ, FOXK, FOXL, FOXM, FOXN, FOXO, FOXP, FOXQ, FOXR, and FOXS.

In some cases, the one or more target genes can comprise an erythroblast transformation specific (ETS) gene. ETS are transcription factors unique to animals and are implicated in tissue development. Non-limiting examples of ETS genes can include ELF1, ELF2 (NERF) ELF4 (MEF) GABPα, ERG, FLI1, FEV, ERF (PE2), ETV3 (PE1), ELF3 (ESE1/ESX), ELF5 (ESE2), ESE3 (EHF), ETS1, ETS2, SPDEF (PDEF/PSE), ETV4 (PEA3/E1AF), ETV5 (ERM), ETV1 (ER81), ETV2 (ER71), SPI1 (PU.1), SPIB, SPIC, ELK1, ELK4 (SAP1), ELK3 (NET/SAP2), ETV6 (TEL), and ETV7 (TEL2).

In some cases, the one or more target genes can comprise a collagen. Collagens are fibrous proteins and are the major elements of skin, bone, tendon, cartilage, blood vessels, and teeth. Collagens form insoluble fibers of high tensile strength. Non-limiting examples of collagen genes can include COLIA1, COLIA2, COL2A1, COL3A1, COL4A1, COL4A2, COL4A3, COL4A4, COL4A5, COL4A6, COL5A1, COL5A2, COL5A3, COL6A1, COL6A2, COL6A3, COL6A4P1, COL6A4P2, COL6A5, COL6A6, COL7A1, COL8A1, COL8A2, COL9A1, COL9A2, COL9A3, COL10A1, COL11A1, COL11A2, COL12A1, COL13A1, COL14A1, COL15A1, COL16A1, COL17A1, COL18A1, COL19A1, COL20A1, COL21A1, COL22A1, COL23A1, COL24A1, COL25A1, COL26A1, COL27A1, and COL28A1.

In some cases, the one or more target genes can comprise a homeobox gene. Homeobox genes are genes that regulate, for example, large-scale anatomical features in the early stages of embryonic development. Types of homeobox genes include HOX genes, LIM genes, PAX genes, POU genes, CERS genes, HNF genes, SINE genes, CUT genes, ZF genes, paraHOX genes, DLX genes, TALE genes, PRD genes, and NKL genes. Non-limiting examples of homeobox genes can include HOXA1, HOXA2, HOXA3, HOXA4, HOXA5, HOXA6, HOXA7, HOXA9, HOXA10, HOXA11, HOXA13, HOXB1, HOXB2, HOXB3, HOXB4, HOXB5, HOXB6, HOXB7, HOXB8, HOXB9, HOXB13, HOXC4, HOXC5, HOXC6, HOXC8, HOXC9, HOXC10, HOXC11, HOXC12, HOXC13, HOXD1, HOXD3, HOXD4, HOXD8, HOXD9, HOXD10, HOXD11, HOXD12, HOXD13, CDX1, CDX2, CDX4, GSX1, GSX2, PDX1, EVX1, EVX2, GBX1, GBX2, MEOX1, MEOX2, MNX1, DLX1, DLX2, DLX3, DLX4, DLX5, DLX6, IRX1, IRX2, IRX3, IRX4, IRX5, IRX6, MEIS1, MEIS2, MEIS3, MKX, PBX1, PBX2, PBX3, PBX4, PKNOX1, PKNOX2, TGIF1, TGIF2, TGIF2LX, TGIF2LY, ISL1, ISL2, LHX1, LHX2, LHX3, LHX4, LHX5, LHX6, LHX8, LHX9, LMX1A, LMX1B, HDX, POUIF1, POU2F1, POU2F2, POU2F3, POU3F1, POU3F2, POU3F3, POU3F4, POU4F1, POU4F2, POU4F3, POU5F1, POU5F1P1, POU5F1P4, POU5F2, POU6F1, POU6F2, LASS2, LASS3, LASS4, LASS5, LASS6, HMBOX1, HNF1A, HNF1B, SIX1, SIX2, SIX3, SIX4, SIX5, SIX6, ONECUT1, ONECUT2, ONECUT3, CUX1, CUX2, SATB1, SATB2, ADNP, ADNP2, TSHZ1, TSHZ2, TSHZ3, ZEB1, ZEB2, ZFHX2, ZFHX3, ZFHX4, ZHX1, HOMEZ, ALX1 (CARTI), ALX3, ALX4, ARGFX, ARX, DMBX1, DPRX, DRGX, DUXA, DUXB, DUX (1, 2, 3, 4, 4c, 5), ESXI, GSC, GSC2, HESX1, HOPX, ISX, LEUTX, MIXLI, NOBOX, OTP, OTX1, OTX2, CRX, PAX2, PAX3, PAX4, PAX5, PAX6, PAX7, PAX8, PHOX2A, PHOX2B, PITX1, PITX2, PITX3, PROPI, PRRX1, PRRX2, RAX, RAX2, RHOXF1, RHOXF2/2B, SEBOX, SHOX, SHOX2, TPRX1, UNCX, VSX1, VSX2, BARHL1, BARHL2, BARX1, BARX2, BSX, DBX1, DBX2, EMX1, EMX2, EN1, EN2, HHEX, HLX1, LBX1, LBX2, MSX1, MSX2, NANOG, NOTO, TLX1, TLX2, TLX3, TSHZ1, TSHZ2, TSHZ3, VAX1, VAX2, VENTX, NKX2-1, NKX2-4, NKX2-2, NKX2-8, NKX3-1, NKX3-2, NKX2-3, NKX2-5, NKX2-6, HMX1, HMX2, HMX3, NKX6-1, NKX6-2, and NKX6-3.

In some cases, the one or more target genes can comprise a GATA gene. GATA genes are transcription factors characterized by their ability to bind to the DNA sequence “GATA.” Non-limiting examples of GATA genes can include GATA1, GATA2, GATA3, GATA4, GATA5, and GATA6.

In some cases, use of the heterologous genetic circuit as disclosed herein can be used to differentiate stem cells (e.g., pluripotent stem cells (PSC), erythro-myeloid progenitor (EMP) cells, mesogenic progenitor cells, hematopoietic progenitor cells) into hematopoietic cells whereby at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, or at least about 100% of the resulting cells generated by using the heterologous genetic circuit as disclosed herein are the target cell type. The target cell type may include T cells, natural killer cells, B cells, dendritic cells, macrophages, or other hematopoietic cells.

In some cases, use of the heterologous genetic circuit as disclosed herein can be used to differentiate stem cells (e.g., pluripotent stem cells (PSC), erythro-myeloid progenitor (EMP) cells, mesogenic progenitor cells, hematopoietic progenitor cells) into immune cells (T cells, NK cells, etc.), e.g., in absence of one, two, or all of feeder cells, serum, and exogenous Notch. Using the heterologous genetic circuit as disclosed herein can generate at least about 1×10⁴, at least about 2×10⁴, at least about 5×10⁴, at least about 1×10⁵, at least about 2×10⁵, at least about 5×10⁵, at least about 1×10⁶, at least about 2×10⁶, at least about 5×10⁶, at least about 1×10⁷, at least about 2×10⁷, at least about 5×10⁷, at least about 1×10⁸, at least about 2×10⁸, at least about 5×10⁸, at least about 1×10⁹, at least about 2×10⁹, at least about 5×10⁹, at least about 1×10¹⁰, at least about 2×10¹⁰, at least about 5×10¹⁰, at least about 1×10¹⁵, at least about 2×10¹⁵, at least about 5×10¹⁵, or more T cells from at most about 1×10⁶, at most about 9×10⁵, at most about 8×10⁵, at most about 7×10⁵, at most about 6×10⁵, at most about 5×10⁵, at most about 4×10⁵, at most about 3×10⁵, at most about 2×10⁵, at most about 1×10⁵, at most about 5×10⁴, at most about 2×10⁴, at most about 1×10⁴, or fewer EMP cells (e.g., human EMP cells).

Such generation of T cells by using the heterologous genetic circuit as disclosed herein can be achieved within the span of at most about 60 days, at most about 55 days, at most about 50 days, at most about 45 days, at most about 40 days, at most about 35 days, at most about 30 days, at most about 25 days, at most about 20 days, at most about 15 days, at most about 10 days, at most about 5 days, or less. The resulting T cells generated by using the heterologous genetic circuit as disclosed herein can induce at least about 40%, at least about 45%, at least about 50%, at least about 55%, at least about 60%, at least about 65%, at least about 70%, at least about 75%, at least about 80%, at least about 85%, at least about 90%, at least about 95%, at least about 99%, or more of target cells (e.g., cancer cells) when ascertained (e.g., in vitro, in vivo) in effector to target (E:T) ratio of at least about 1:3, at least about 1:4, at least about 1:5, at least about 1:6, at least about 1:7, at least about 1:8, at least about 1:9, at least about 1:10, at least about 1:12, at least about 1:15, at least about 1:20, at least about 1:30, at least about 1:40, at least about 1:50, or less (e.g., the E:T ratio of 1:100 is less than the E:T ratio of 1:50). When injected into mice, the resulting T cells generated by using the heterologous genetic circuit as disclosed herein can extend mouse lifespan at least about 30%, at least about 40%, at least about 50%, at least about 60%, at least about 70%, at least about 80%, at least about 90%, at least about 100%, or more.

In some cases, the T cells generated through this method have superior killing function to T cells obtained via directed differentiation. The T cells generated using the provided methods can have killing function at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, or at least about 500% greater than the killing function of T cells obtained via directed differentiation. Alternatively, or in addition to, the T cells generated through this method may be equivalent killing function as compared to primary peripheral blood derived T cells.

In some cases, generated T cells other than primary peripheral derived T cells have killing abilities similar to primary peripheral derived T cells as measured through CAR activity. T cell types can include, but are not limited to, gamma-delta T cells, NKT cells, and MAIT cells. In some cases, use of the heterologous genetic circuit as disclosed herein can be used to obtain a cell with an unnatural phenotype (e.g., a non-T cell that has killing abilities).

In some cases, use of the heterologous genetic circuit in the stem cells (e.g., pluripotent stem cells (PSC), erythro-myeloid progenitor (EMP) cells, mesogenic progenitor cells, hematopoietic progenitor cells) can induce the stem cells to differentiate into immune cells in absence of feeder cells. In some cases, use of the heterologous genetic circuit in the stem cells (e.g., pluripotent stem cells (PSC), erythro-myeloid progenitor (EMP) cells, mesogenic progenitor cells, hematopoietic progenitor cells) can induce the stem cells to differentiate into immune cells in absence of serum. In some cases, use of the heterologous genetic circuit in the stem cells (e.g., pluripotent stem cells (PSC), erythro-myeloid progenitor (EMP) cells, mesogenic progenitor cells, hematopoietic progenitor cells) can induce the stem cells to differentiate into immune cells in absence of exogenous Notch. In some cases, use of the heterologous genetic circuit as disclosed herein can be used to differentiate stem cells (e.g., pluripotent stem cells (PSC), erythro-myeloid progenitor (EMP) cells, mesogenic progenitor cells, hematopoietic progenitor cells) into T cells e.g., in the absence of one or both embryoid bodies and serum. The resulting T cells generated by using the heterologous genetic circuit as disclosed herein are at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90% or at least about 100% of the total resultant cell population. In some cases, use of the heterologous genetic circuit as disclosed herein can be used to differentiate stem cells (e.g., pluripotent stem cells (PSC), erythro-myeloid progenitor (EMP) cells, mesogenic progenitor cells, hematopoietic progenitor cells) into natural killer cells e.g., in the absence of one or both embryoid bodies and serum. The resulting natural killer cells generated by using the heterologous genetic circuit as disclosed herein are at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90% or at least about 100% of the total resultant cell population.

In some cases, use of the heterologous genetic circuit as disclosed herein can be used to differentiate stem cells (e.g., pluripotent stem cells (PSC), erythro-myeloid progenitor (EMP) cells, mesogenic progenitor cells, hematopoietic progenitor cells) into B cells. The resulting B cells generated by using the heterologous genetic circuit as disclosed herein are at least about 5%, at least about 6%, at least about 7%, at least about 8%, at least about 9%, at least about 10%, at least about 15%, at least about 20%, at least about 30%, at least about 40%, at least about 50%, at least about 60%, at least about 70%, at least about 80%, at least about 90% or at least about 100% of the total resultant cell population after differentiation.

In some cases, use of the heterologous genetic circuit as disclosed herein can be used to differentiate stem cells (e.g., pluripotent stem cells (PSC), erythro-myeloid progenitor (EMP) cells, mesogenic progenitor cells, hematopoietic progenitor cells) into dendritic cells. The resulting dendritic cells generated by using the heterologous genetic circuit as disclosed herein are at least about 40%, at least about 50%, at least about 60%, at least about 70%, at least about 80%, at least about 90% or at least about 100% of the total resultant cell population after differentiation.

In some cases, use of the heterologous genetic circuit as disclosed herein can be used to differentiate stem cells (e.g., pluripotent stem cells (PSC), erythro-myeloid progenitor (EMP) cells, mesogenic progenitor cells, hematopoietic progenitor cells) into macrophages. The resulting macrophages generated by using the heterologous genetic circuit as disclosed herein are similar to the naïve cell state in their ability to detect, phagocytose, and destroy harmful cells as measured by their ability to act as a surrogate for a CAR or by their ability to kill cancerous cells.

In some cases, a target gene may be subjected to at least two distinct modulations comprising a first modulation and a second modulation. Timing of the first modulation and the second modulation can be controlled (e.g., as predetermined by the design of the heterologous genetic circuit). For example, the onset of the second modulation (e.g., by at least a portion of the second gate unit, such as the second gene regulation moiety) can occur subsequent to the onset of the first modulation (e.g., by at least a portion of the first gate unit, such as the first gene regulating moiety) by at least about 1 second, at least about 2 seconds, at least about 3 seconds, at least about 4 seconds, at least about 5 seconds, at least about 6 seconds, at least about 7 seconds, at least about 8 seconds, at least about 9 seconds, at least about 10 seconds, at least about 20 seconds, at least about 30 seconds, at least about 40 seconds, at least about 50 seconds, at least about 1 minute, at least about 2 minutes, at least about 3 minutes, at least about 4 minutes, at least about 5 minutes, at least about 6 minutes, at least about 7 minutes, at least about 8 minutes, at least about 9 minutes, at least about 10 minutes, at least about 20 minutes, at least about 30 minutes, at least about 40 minutes, at least about 50 minutes, at least about 1 hour, at least about 2 hours, at least about 3 hours, at least about 4 hours, at least about 5 hours, at least about 6 hours, at least about 7 hours, at least about 8 hours, at least about 9 hours, at least about 10 hours, at least about 20 hours, at least about 1 day, at least about 2 days, at least about 3 days, at least about 4 days, at least about 5 days, at least about 6 days, at least about 7 days, at least about 8 days, at least about 9 days, or at least about 10 days. The onset of the second modulation (e.g., by at least a portion of the second gate unit, such as the second gene regulation moiety) can occur subsequent to the onset of the first modulation (e.g., by at least a portion of the first gate unit, such as the first gene regulation moiety) by at most about 10 days, at most about 9 days, at most about 8 days, at most about 7 days, at most about 6 days, at most about 5 days, at most about 4 days, at most about 3 days, at most about 2 days, at most about 1 day, at most about 20 hours, at most about 10 hours, at most about 9 hours, at most about 8 hours, at most about 7 hours, at most about 6 hours, at most about 5 hours, at most about 4 hours, at most about 3 hours, at most about 2 hours, at most about 1 hours, at most about 50 minutes, at most about 40 minutes, at most about 30 minutes, at most about 20 minutes, at most about 10 minutes, at most about 9 minutes, at most about 8 minutes, at most about 7 minutes, at most about 6 minutes, at most about 5 minutes, at most about 4 minutes, at most about 3 minutes, at most about 2 minutes, at most about 1 minutes, at most about 50 seconds, at most about 40 seconds, at most about 30 seconds, at most about 20 seconds, at most about 10 seconds, at most about 9 seconds, at most about 8 seconds, at most about 7 seconds, at most about 6 seconds, at most about 5 seconds, at most about 4 seconds, at most about 3 seconds, at most about 2 seconds, or at most about 1 second.

In some cases, a number of gate units that need to be activated (e.g., sequentially activated) between the activation of the first modulation by the first gate unit and the later activation of the second modulation by the second gate unit can at least in part determine (e.g., substantially determine) the timing between the first modulation and the second modulation. Upon activation of the first modulation of the target gene by the first gate unit, at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more additional gate units may need to be activated (e.g., sequentially activated) to activate the second gate unit for inducing the second modulation. Upon activation of the first modulation of the target gene by the first gate unit, at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 additional gate units may need to be activated (e.g., sequentially activated) to activate the second gate unit for inducing the second modulation.

The outcome of a cell can comprise the regulation of a plurality of target genes. For example, the outcome can comprise the regulation of at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more target genes. The outcome can comprise the regulation of at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 target gene(s). Each gene that is disclosed herein can be subjected to at least about 1, at least about 2, at least about 3, at least about 4, at least about 5, at least about 6, at least about 7, at least about 8, at least about 9, at least about 10, at least about 15, at least about 20, at least about 30, at least about 40, at least about 50, or more modulations. Each gene that is disclosed herein can be subjected to at most about 50, at most about 40, at most about 30, at most about 20, at most about 15, at most about 10, at most about 9, at most about 8, at most about 7, at most about 6, at most about 5, at most about 4, at most about 3, at most about 2, or at most about 1 modulation(s). One or more modulations of a target gene (e.g., an endogenous gene), as induced by the heterologous genetic circuit of the present disclosure, may be an artificial modulation (or a heterologous modulation) that may otherwise not occur in the cell in absence of (i) the heterologous genetic circuit and/or (ii) the activating moiety of the heterologous genetic circuit.

The plurality of gate units can operate sequentially (e.g., each of the plurality of gate units is activated in a sequential manner). For example, a gate unit of the plurality to be activated to activate a subsequent gate unit of the plurality. Sequential operation of the gate units can be linear. Alternatively, sequential operation of the gate units can route back on one another as inputs to form a loop. For example, a plurality of the gate units can induce a feedback loop such as a positive feedback loop or a negative feedback loop.

In some embodiments of any one of the systems disclosed herein, the first gate unit can comprise a first gene regulating moiety that can be activatable to exhibit specific binding to the target gene to induce a first distinct modulation. Alternatively or in addition to, the first gate unit can comprise a first gene regulating moiety that can be activatable to exhibit non-specific binding to the target gene to induce the first distinct modulation.

The first distinct modulation can induce a change (e.g., increase or decrease) in the expression and/or activity level of the target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, or more. The first distinct modulation can induce a change (e.g., increase or decrease in the expression and/or activity level of the target gene by at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, at most about 0.1%, or less.

The first distinct modulation as disclosed herein (e.g., induced by the first gate unit) can induce a change (e.g., increase or decrease) in the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. The first distinct modulation can induce a change (e.g., increase or decrease) in the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

Subsequently, a second distinct modulation as disclosed herein (e.g., induced by the second gate unit) can induce an additional change (e.g., increase, decrease, or selective attenuation) in the expression and/or activity level of the target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, or at least about 1,000,000%. The second distinct modulation can induce an additional change (e.g., increase or decrease) in the expression and/or activity level of the target gene by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.1%.

The additional change via the second distinct modulation can induce an additional change (e.g., increase or decrease) in the expression and/or activity level of the target gene by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. The second distinct modulation can induce an additional change (e.g., increase or decrease) in the expression and/or activity level of the target gene by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

The additional change via the second distinct modulation can occur when the expression and/or activity level of the target gene reaches a target level via action of the first distinct modulation, e.g., by design of the heterologous genetic circuit.

The additional change via the second distinct modulation can occur when the expression and/or activity level of the target gene is changed (e.g., increased or decreased) via action of the first distinct modulation by at least or up to about 0.1-fold, at least or up to about 0.2-fold, at least or up to about 0.3-fold, at least or up to about 0.4-fold, at least or up to about 0.5-fold, at least or up to about 0.6-fold, at least or up to about 0.7-fold, at least or up to about 0.8-fold, at least or up to about 0.9-fold, at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold, as compared to a control expression and/or activity level. The additional change via the second distinct modulation can occur when the expression and/or activity level of the target gene is changed (e.g., increased or decreased) via action of the first distinct modulation by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, at most or less than about 1-fold, at most or less than about 0.9-fold, at most or less than about 0.8-fold, at most or less than about 0.7-fold, at most or less than about 0.6-fold, at most or less than about 0.5-fold, at most or less than about 0.4-fold, at most or less than about 0.3-fold, at most or less than about 0.2-fold, at most or less than about 0.1-fold, as compared to a control expression and/or activity level.

Alternatively, or in addition to, a second distinct modulation as disclosed herein (e.g., induced by the second gate unit) can induce a change (e.g., increase or decrease) in the expression and/or activity level of an additional target gene by at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, at least about 60%, at least about 70%, at least about 80%, at least about 90%, at least about 100%, at least about 200%, at least about 300%, at least about 400%, at least about 500%, at least about 600%, at least about 700%, at least about 800%, at least about 900%, at least about 1,000%, at least about 2,000%, at least about 3,000%, at least about 4,000%, at least about 5,000%, at least about 6,000%, at least about 7,000%, at least about 8,000%, at least about 9,000%, at least about 10,000%, at least about 100,000%, or at least about 1,000,000%. The second distinct modulation can induce a change (e.g., increase or decrease) in the expression and/or activity level of the additional target gene by at most about 1,000,000%, at most about 100,000%, at most about 9,000%, at most about 8,000%, at most about 7,000%, at most about 6,000%, at most about 5,000%, at most about 4,000%, at most about 3,000%, at most about 2,000%, at most about 1,000%, at most about 900%, at most about 800%, at most about 700%, at most about 600%, at most about 500%, at most about 400%, at most about 300%, at most about 200%, at most about 100%, at most about 90%, at most about 80%, at most about 70%, at most about 60%, at most about 50%, at most about 40%, at most about 30%, at most about 20%, at most about 10%, at most about 9%, at most about 8%, at most about 7%, at most about 6%, at most about 5%, at most about 4%, at most about 3%, at most about 2%, at most about 1%, at most about 0.9%, at most about 0.8%, at most about 0.7%, at most about 0.6%, at most about 0.5%, at most about 0.4%, at most about 0.3%, at most about 0.2%, or at most about 0.1%. A cell can comprise a prokaryotic cell, a eukaryotic cell, or an artificial cell.

A cell (e.g., an initial cell to be modified into the engineered cell as disclosed herein, a final cell product generated from the engineered cell as disclosed herein, etc.) can comprise a muscle cell, an immune cell, a neuron, an osteoblast, an endothelial cell, an mesenchymal cell, an epithelial cell, a stem cell, an secretory cell, a blood cell, a germ cell, a nurse cell, a storage cell, an enteroendocrine cell, a pituitary cell, a neurosecretory cell, a duct cell, an odontoblast, a cementoblast, a glial cell, or an interstitial cell.

Non-limiting examples of such cell can include lymphoid cells, such as B cell, T cell (Cytotoxic T cell, Natural Killer T cell, Regulatory T cell, T helper cell), Natural killer cell, cytokine induced killer (CIK) cells (see e.g. US20080241194); myeloid cells, such as granulocytes (Basophil granulocyte, Eosinophil granulocyte, Neutrophil granulocyte/Hypersegmented neutrophil), Monocyte/Macrophage, Red blood cell (Reticulocyte), Mast cell, Thrombocyte/Megakaryocyte, Dendritic cell; cells from the endocrine system, including thyroid (Thyroid epithelial cell, Parafollicular cell), parathyroid (Parathyroid chief cell, Oxyphil cell), adrenal (Chromaffin cell), pineal (Pinealocyte) cells; cells of the nervous system, including glial cells (Astrocyte, Microglia), Magnocellular neurosecretory cell, Stellate cell, Boettcher cell, and pituitary (Gonadotrope, Corticotrope, Thyrotrope, Somatotrope, Lactotroph); cells of the Respiratory system, including Pneumocyte (Type I pneumocyte, Type II pneumocyte), Clara cell, Goblet cell, Dust cell; cells of the circulatory system, including Myocardiocyte, Pericyte; cells of the digestive system, including stomach (Gastric chief cell, Parietal cell), Goblet cell, Paneth cell, G cells, D cells, ECL cells, I cells, K cells, S cells; enteroendocrine cells, including enterochromaffm cell, APUD cell, liver (Hepatocyte, Kupffer cell), Cartilage/bone/muscle; bone cells, including Osteoblast, Osteocyte, Osteoclast, teeth (Cementoblast, Ameloblast); cartilage cells, including Chondroblast, Chondrocyte; skin cells, including Trichocyte, Keratinocyte, Melanocyte (Nevus cell); muscle cells, including Myocyte; urinary system cells, including Podocyte, Juxtaglomerular cell, Intraglomerular mesangial cell/Extraglomerular mesangial cell, Kidney proximal tubule brush border cell, Macula densa cell; reproductive system cells, including Spermatozoon, Sertoli cell, Leydig cell, Ovum; and other cells, including Adipocyte, Fibroblast, Tendon cell, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet stratified barrier epithelial cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining urinary bladder and urinary ducts), Exocrine secretory epithelial cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion). Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (hydrochloric acid secretion), Pancreatic acinar cell (bicarbonate and digestive enzyme secretion), Paneth cell of small intestine (lysozyme secretion), Type II pneumocyte of lung (surfactant secretion), Clara cell of lung, Hormone secreting cells, Anterior pituitary cells, Somatotropes, Lactotropes, Thyrotropes, Gonadotropes, Corticotropes, Intermediate pituitary cell, Magnocellular neurosecretory cells, Gut and respiratory tract cells, Thyroid gland cells, thyroid epithelial cell, parafollicular cell, Parathyroid gland cells, Parathyroid chief cell, Oxyphil cell, Adrenal gland cells, chromaffin cells, Ley dig cell of testes, Theca interna cell of ovarian follicle, Corpus luteum cell of ruptured ovarian follicle, Granulosa lutein cells, Theca lutein cells, Juxtaglomerular cell (renin secretion), Macula densa cell of kidney, Metabolism and storage cells, Barrier function cells (Lung, Gut, Exocrine Glands and Urogenital Tract), Kidney, Type I pneumocyte (lining air space of lung), Pancreatic duct cell (centroacinar cell), Nonstriated duct cell (of sweat gland, salivary gland, mammary gland, etc.), Duct cell (of seminal vesicle, prostate gland, etc.), Epithelial cells lining closed internal body cavities, Ciliated cells with propulsive function, Extracellular matrix secretion cells, Contractile cells; Skeletal muscle cells, stem cell, Heart muscle cells, Blood and immune system cells, Erythrocyte (red blood cell), Megakaryocyte (platelet precursor), Monocyte, Connective tissue macrophage (various types), Epidermal Langerhans cell, Osteoclast (in bone), Dendritic cell (in lymphoid tissues), Microglial cell (in central nervous system), Neutrophil granulocyte, Eosinophil granulocyte, Basophil granulocyte, Mast cell, Helper T cell, Suppressor T cell, Cytotoxic T cell, Natural Killer T cell, B cell, Natural killer cell, Reticulocyte, Stem cells and committed progenitors for the blood and immune system (various types), Pluripotent stem cells, Totipotent stem cells, Induced pluripotent stem cells, adult stem cells, Sensory transducer cells, Autonomic neuron cells, Sense organ and peripheral neuron supporting cells, Central nervous system neurons and glial cells, Lens cells, Pigment cells, Melanocyte, Retinal pigmented epithelial cell, Germ cells, Oogonium/Oocyte, Spermatid, Spermatocyte, Spermatogonium cell (stem cell for spermatocyte), Spermatozoon, Nurse cells, Ovarian follicle cell, Sertoli cell (in testis), Thymus epithelial cell, Interstitial cells, and Interstitial kidney cells.

A stem cell can comprise an induced pluripotent stem cell (iPSC), a pluripotent stem cell (PSC), an embryonic stem cell (ESC), a mesenchymal stem cell (MSC), a erythro-myeloid progenitor (EMP) cell, a mesogenic progenitor cell, a hematopoietic stem cell (HSC), a hematopoietic progenitor cell, a muscle stem cell, a neural stem cell, an epithelial stem cell, an epidermal stem cell, a mammary stem cell, an intestinal stem cell, a neural crest stem cell, or a testicular stem cell.

Various aspects of the present disclosure provide engineered cells that are programmed to induce a desired expression and/or activity level (or profile thereof) of one or more target genes in a cell.

In some embodiments, the engineered cell (e.g., the engineered muscle cell, the engineered immune cell) of the present disclosure can be generated from an isolated stem cell (e.g., isolate ESCs, iPSCs, EMPs, etc.). The heterologous genetic circuit and/or its components (e.g. gate units, gate moieties, activating moieties, etc.), as disclosed herein, can be introduced during any stage (or cellular state) between and including (a) the isolated stem cell and (b) the differentiated immune cell state thereof (e.g. a terminally differentiated immune cell state, such as a terminally differentiated T cell.

In some cases, the engineered cells T cells can be derived from EMPs, and the heterologous genetic circuit and/or its components (e.g., the heterologous gate units, the heterologous activating moieties, the heterologous gate moieties, etc.) can be introduced to the cell at (A) the EMP state, (B) the hematopoietic stem cell state, (C) the T cell state or (D) any other intermediary cell states. The heterologous genetic circuit and/or its components (e.g., the heterologous gate units, the heterologous activating moieties, the heterologous gate moieties, etc.) can be introduced to the cell once during one of (A), (B), (C), or (D). Alternatively, the heterologous genetic circuit and/or its components (e.g., the heterologous gate units, the heterologous activating moieties, the heterologous gate moieties, etc.) can be introduced to the cell multiple times during two, three, or all of (A), (B), (C), or (D).

The engineered cell (e.g., the engineered T cell) of the present disclosure can be used (e.g., administered) to treat a subject in need thereof. The subject can have or can be suspected of having a condition, such as a disease (e.g., cancer). A cell (e.g., a stem cell or a differentiated cell) can be obtained from the subject and such cell can be cultured ex vivo and genetically modified to generate any subject engineered cell (e.g. any immune cell) as disclosed herein. Subsequently, the engineered immune cell can be administered to the subject for adaptive immunotherapy. Thus, the engineered cell can be autologous to the subject in need thereof. Alternatively, the engineered cell can be allogeneic to the subject (e.g., allogeneic stem cell transplantation, allogeneic adoptive immunotherapy, etc.).

A cell or construct of the present disclosure can be used (e.g., administered) in a pharmaceutical formulation. A pharmaceutical formulation can further comprise an additional therapeutic agent. An additional therapeutic agent can comprise a chemotherapeutic agent, an immunosuppressing agent, and/or an antibiotic agent.

A chemotherapeutic agent, also known as an antineoplastic agent, is a type of cancer treatment used to directly or indirectly inhibit the growth and proliferation of cancer cells. A chemotherapeutic agent can be an alkylating agent (e.g., an oxazaphosphorine, a nitrogen mustard, an imidazotetrazine, a nitrosourea, an alkyl sulfonate, a hydrazine, or a platinum based agent), an antimetabolite (e.g., an antifolate, a pyrimidine antagonist, a purine antagonist, or a ribonuclease reductase inhibitor), a topoisomerase inhibitor (e.g., a topoisomerase I inhibitor or a topoisomerase II inhibitor), an antibiotic (e.g., a bleomycin, an actinomycin D, an anthracycline, or a mitomycin), a mitotic inhibitor (e.g., a vinca alkaloid, a taxane, or a nontaxane microtubule inhibitor), a protein kinase inhibitor (e.g., a tyrosine kinase inhibitor, a MEK inhibitor, a CDK inhibitor), a proteasome inhibitor, or a PARP inhibitor.

An immunosuppressing agent is a an agent that decreases an immune response. Non-limiting examples of immunosuppressing agents can include steroids (e.g., prednisone, methylprednisolone, dexamethasone), colchicine, hydroxychloroquine, sulfasalazine, dapsone, methotrexate, mycophenolate mofetil, azathioprine, anti-IL-1 biologics, anti-TNF biologics, anti-IL-6 biologics, B cell growth factor targeting biologics, T cells, cytokines, or JAK inhibitors.

An antibiotic agent is a an agent that destroys or inhibits the growth of microorganisms. Non-limiting examples of antibiotic agents can include tetracycline, oxytetracycline, metacycline, doxycycline, minocycline, erythromycin, lincomycin, penicillin G, clindamycin, kanamycin, chloramphenicol, fradiomycin, streptomycin, norfloxacin, ciprofloxacin, ofloxacin, grepafloxacin, levofloxacin, sparfloxacin, ampicillin, carbenicillin, methicillin, cephalosporins, vancomycin, bacitracin, gentamycin, fusidic acid, ciprofloxin and other quinolones, erythromycin, gentamicin, sulfonamides, trimethoprim, dapsone, isoniazid, teicoplanin, avoparcin, synercid, virginiamycin, piperacillin, ticarcillin, cefepime, cefpirome, rifampicin, pyrazinamide, enrofloxacin, amikacin, netilmycin, imipenem, meropenem, inezolidcefuroxime, ceftriaxone, cefadroxil, cefazoline, ceftazidime, cefotaxime, roxithromycin, cefaclor, cefalexin, cefoxitin, amoxicillin, co-amoxiclav, mupirocin, cloxacillin, and co-trimoxazole.

A pharmaceutical formulation can further comprise an excipient. An excipient can be a buffer, a carrier, a stabilizer, a solubilizer, a filler, a preservative, a dilutant, a vehicle, a detergent, a salt, a peptide, a surfactants, an oligosaccharide, an amino acid, an adjuvant, a carbohydrate, and/or a bulking agent.

The engineered cells as disclosed herein can be administered to the subject prior to, concurrently with, or subsequent to activation of the heterologous genetic circuit(s) in the engineered stem cells. For example, the engineered cells can be activated subsequent to being administered into the subject, e.g., by administering to the subject an activator of the heterologous genetic circuit(s).

The subject can be treated (e.g., administered with) a population of engineered cells (e.g., engineered T cells) of the present disclosure for at least or up to about 1 dose, at least or up to about 2 doses, at least or up to about 3 doses, at least or up to about 4 doses, at least or up to about 5 doses, at least or up to about 6 doses, at least or up to about 7 doses, at least or up to about 8 doses, at least or up to about 9 doses, or at least or up to about 10 doses. Alternatively, or in addition to, the subject can be treated (e.g., administered with) a population of engineered cells (e.g., engineered T cells) of the present disclosure for 1 week, 2 weeks, 3 weeks, 4 weeks, 5 weeks, 6, weeks, 2 months, 3 months, 4 months, 5 months, 6 months, 7 months, 8 months, 9 months, 10 months, 11 months, 1 year, 2 years, 3 years, 4 years, 5 years, 6 years, 7 years, 8 years, 9 years, 10 years, 15 years, 20 years, 30 years, 40 years, 50 years, 60 years, 70 years, 80 years, 90 years, or 100 years.

Any one of the methods disclosed herein can be utilized to treat a target cell, a target tissue, a target condition, or a target disease of a subject.

Non-limiting examples of the target tissue can include cells, for example immune cells (e.g., lymphocytes including T cells and NK cells), can be obtained from a subject. Non-limiting examples of subjects include humans, dogs, cats, mice, rats, and transgenic species thereof. Examples of samples from a subject from which cells can be derived include, without limitation, skin, heart, lung, kidney, bone marrow, breast, pancreas, liver, muscle, smooth muscle, bladder, gall bladder, colon, intestine, brain, prostate, esophagus, thyroid, serum, saliva, urine, gastric and digestive fluid, tears, stool, semen, vaginal fluid, interstitial fluids derived from tumorous tissue, ocular fluids, sweat, mucus, earwax, oil, glandular secretions, spinal fluid, hair, fingernails, plasma, nasal swab or nasopharyngeal wash, spinal fluid, cerebral spinal fluid, tissue, throat swab, biopsy, placental fluid, amniotic fluid, cord blood, emphatic fluids, cavity fluids, sputum, pus, microbiota, meconium, breast milk, and/or other excretions or body tissues.

The target disease of the subject can be cancer or tumor. Non-limiting examples of cancer can include cells of cancers including Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloblastoma, Medulloepithelioma, Melanoma, Melanoma, Meningioma, Merkel Cell Carcinoma, Mesothelioma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple Myeloma, Multiple myeloma, Mycosis Fungoides, Mycosis fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neuroblastoma, Neurofibroma, Neuroma, Nodular melanoma, Non-Hodgkin Lymphoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral Cancer, Oral cancer, Oropharyngeal Cancer, Osteosarcoma, Osteosarcoma, Ovarian Cancer, Ovarian cancer, Ovarian Epithelial Cancer, Ovarian Germ Cell Tumor, Ovarian Low Malignant Potential Tumor, Paget's disease of the breast, Pancoast tumor, Pancreatic Cancer, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, Wilms' tumor, and combinations thereof. In some embodiments, the targeted cancer cell represents a subpopulation within a cancer cell population, such as a cancer stem cell. In some embodiments, the cancer is of a hematopoietic lineage, such as a lymphoma. The antigen can be a tumor associated antigen.

The present disclosure also provides a composition comprising the engineered genetic circuit(s) as disclosed herein. The composition can further comprise the actuator of the heterologous genetic circuit(s). The present disclosure also provides a kit comprising the composition. The kit can further comprise the activator(s) of the heterologous genetic circuit(s). The activator(s) can be in the same composition as the engineered cells. Alternatively or in addition to, the activator(s) can be in a different and separate composition from the engineered cells.

### EXAMPLES

### Example 1: Differentiation of T Cells

In this prophetic example, a CRISPR all-at-once method is tested against various heterologous genetic circuits (HGC) to show that the HGCs outperform the CRISPR all-at-once method.

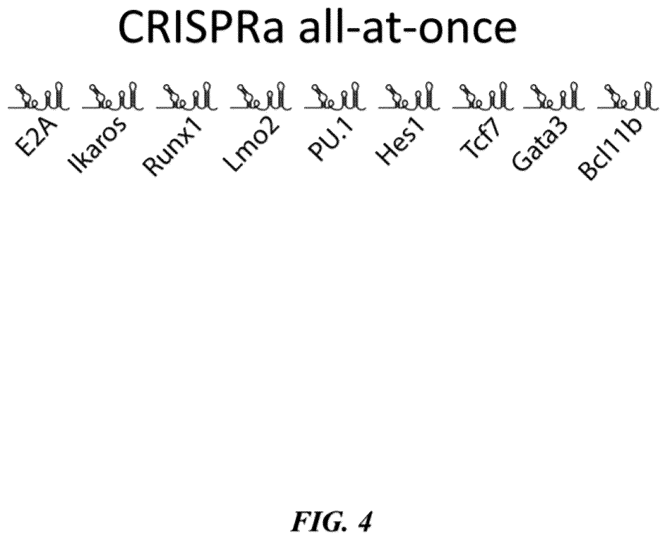

FIG. 4 shows the 9 gRNAs (E2A, Ikaros, Runx1, Lmo2, PU.1, Hes1, Tcf7, Gata3, and Bcl11b) used in the CRISPR all-at-once method. In the CRISPR all-in-one method, the gRNAs are simultaneously added into the cell through nucleofection. The cells are incubated until T-cell differentiation is complete.

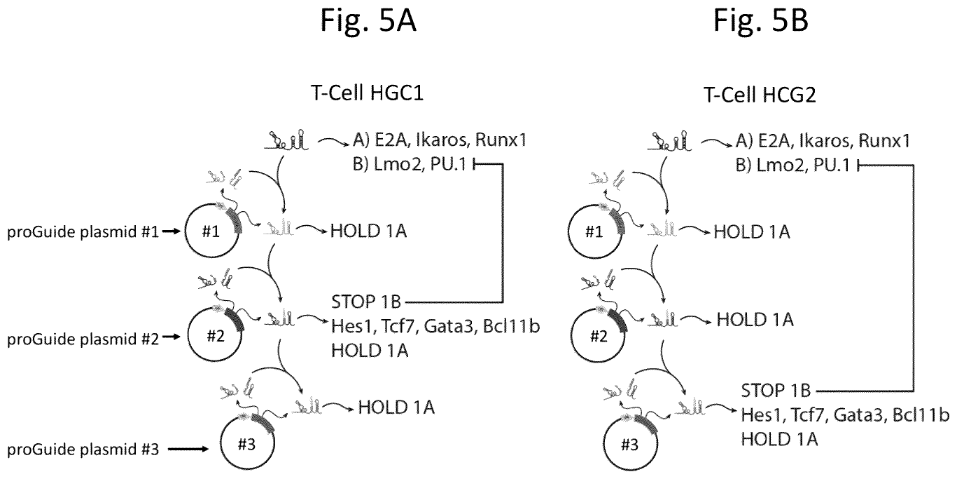

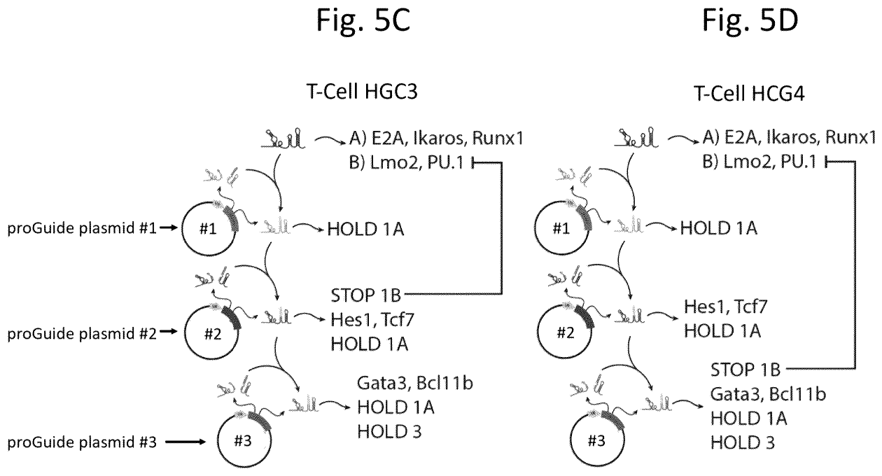

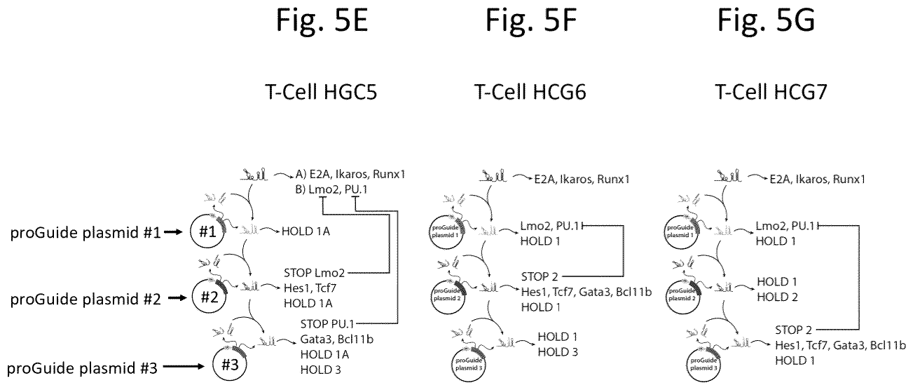

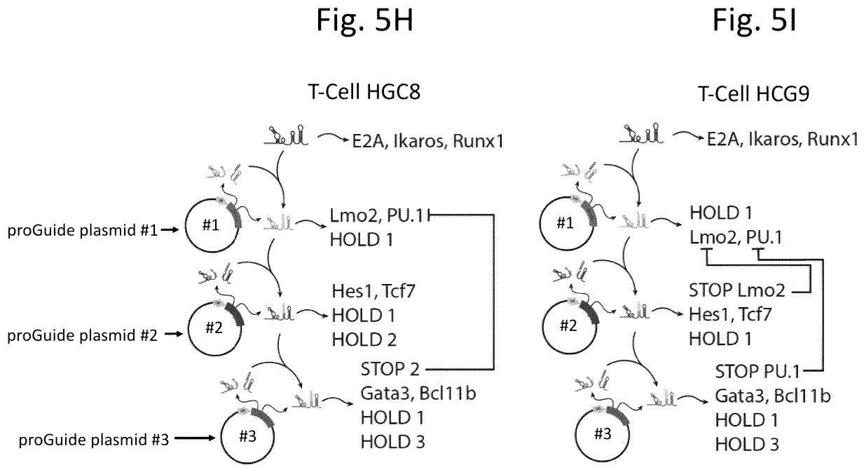

FIG. 5A-5I show the T-Cell HGCs (T-Cell HGC1-9) used in these experiments. In the HGC method, the activating moieties of a HGC are added to a cell through nucleofection. The cells are incubated until T-cell differentiation is complete.

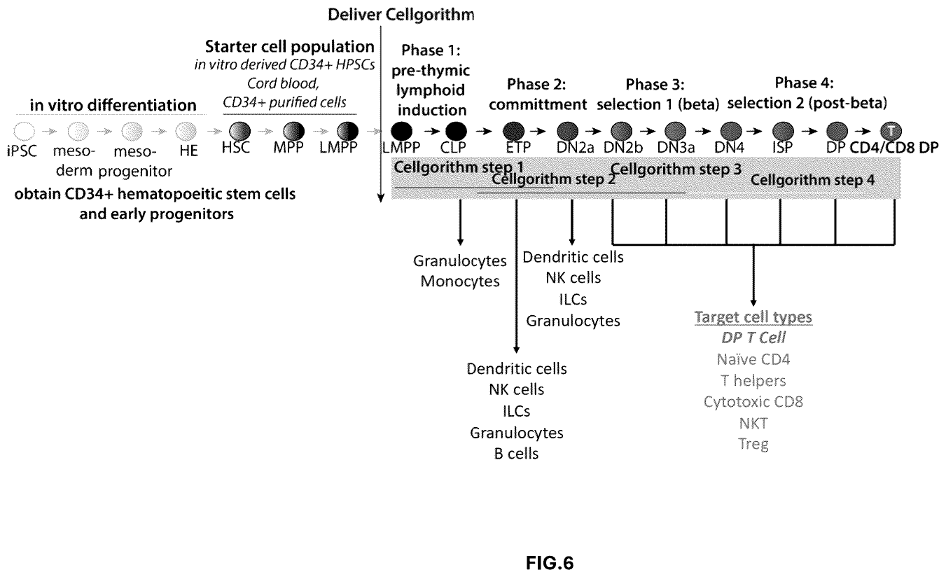

FIG. 5A shows an example of an engineered cell contacted with T-Cell HGC1. This engineered cell is activatable to exhibit the expression and activity profiles of multiple genes associated with T cell differentiation. T-Cell HGC1 comprises four gate units, comprising four different steps. Gate unit 1 of HGC1 activates the genes E2A, Ikaros, Runx1, Lmo2 and PUI and activates gate unit 2. Gate unit 2 of HGC1 only activates gate unit 3 and acts as a hold phase, used as a filler to help with developmental timing. Gate unit 3 of HGC1 activates the genes Hes1, Tcf7, Gata3, and Bcl11b; additionally, it both activates gate unit 4 and negatively modulates the earlier-activated genes Lmo2 and PUI. FIG. 6 depicts how the four Cellgorithm steps work in concert to effect differentiation from starter cells to T cells. Gate unit 1 of T-Cell HGC1 is activatable by an injection that includes a CRISPR sequence tailored to activated gate unit 1. Once the first gate unit is activated, the Cellgorithm proceeds without any outside activation until the heterologous genetic circuit is completed (e.g. Gate unit 4/step 4). The result is an engineered T cell. Other Cellgorithms not shown here function by having the third gate unit reinforce the expression and activity profiles of genes modulated by the first gate unit.

One control cell population is generated using a standard T-cell culturing methods. iPSCs are formed into embryoid bodies and grown for 12 days in media containing BMP-4, VEGF, and bFGF. At day 12, the CD34+ cells are purified out using magnetic beads and are plated on DLL4 coated plates. The cells are grown in media containing TPO, IL-7, SDF, FLT3L, and SCF for 21 days. On day 21, the cells are stimulated with a CD3 antibody in media containing Il-7, IL-2, DXM, allowing for proliferation of mature T cells. Another control T cell population is collected and purified from mouse blood samples.

HGC-generated T-cells can be grown in larger quantities than T-cells generated using the CRISPR all-at-once method. HGC-generated T-cells exhibit normal signal transduction, gene regulation, and proliferation control as compared to innate T-cells collected from blood samples. Additionally, unlike media cultured cells, HGC-generated T-cells may not lose cytotoxic activity over time, have high killing efficiencies (e.g. lower effector to target ratios), longer persistence, and better control and interaction with APCs. Through generation of CD4 cells, the ratio of CD4+ and CD8+ cells can also be controlled, and the persistence and ability of cells to interact with other immune cells that kill like NK cells can be influenced.

It shall be understood that different aspects of the invention can be appreciated individually, collectively, or in combination with each other. Various aspects of the invention described herein may be applied to any of the particular applications disclosed herein. The compositions of matter including compounds of any formulae disclosed herein in the composition section of the present disclosure may be utilized in the method section including methods of use and production disclosed herein, or vice versa.

### Example 2: Differentiation of Hematopoietic Progenitor Cells

In this example, induced pluripotent stem cells (iPSCs) are differentiated into hematopoietic progenitor cells using various heterologous genetic circuits (HGCs).

iPSC Maintenance

Episomal iPSCs (Gibco, CatA18945) were maintained in complete Essential 8TM Flex Medium (Gibco, Cat #A2858501) or StemFlex™ Medium (Gibco, Cat #A3349401) on Vitronectin (Gibco, Cat #A31804)-coated flasks, and passaged to maintain 50-90% confluency using Versene (Gibco, Cat #15040066) to lift cells while maintaining cell clumps.

### Nucleofaction

On Day 0 of the experiment, iPSC cells were collected as a single cell suspension using ACCUTASE™ (Stemcell Technologies, Cat #07920). Cells were resuspended at 1.2e9 cells/ml in P3 Primary Cell Nucleofector™ Solution and 8e5 cells were nucleofected per well in a 96 well nucleocuvette (Lonza, Cat #V4SP-3096) on Lonza's 4D Nucleofector with 96 well shuttle (Cat #AAF-1003B/S) using optimized pulse codes.

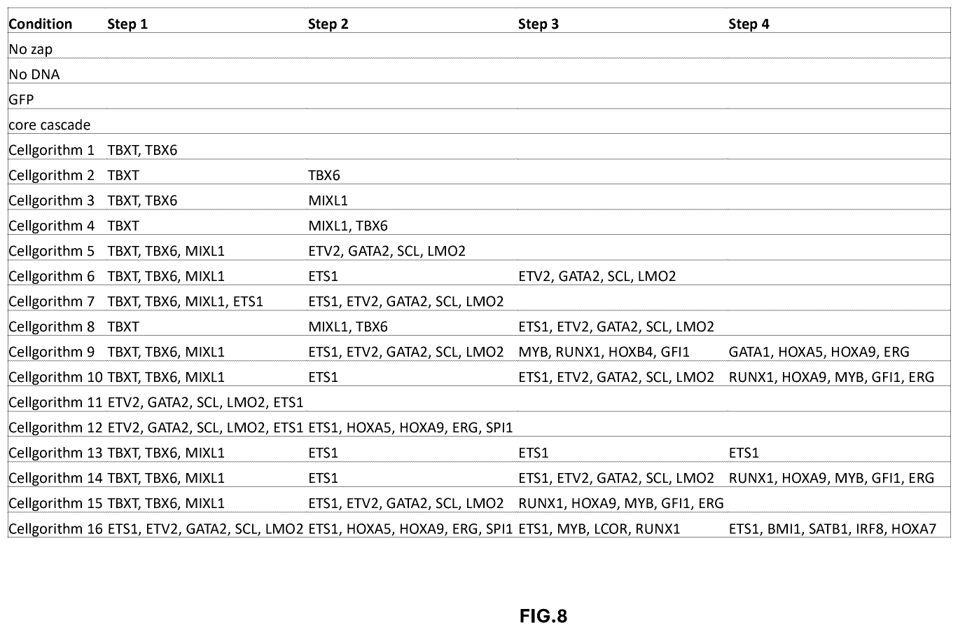

Cells were nucleofected with plasmids encoding Cas9-VPR, the core cascade and the gene-targeting spacers. Cas9-VPR is a Cas-transcriptional modulator system which can be used for both cleavage (e.g., of a gate moiety plasmid, of a gene-regulating moiety plasmid) and non-cleavage gene regulation (e.g., CRISPR activation of a target endogenous gene, CRISPR inhibition of a target endogenous gene) depending on the specific gNA used. The activatable gNAs used are polyT tract only, with a constant core cascade that executes four sequential steps. Nucleotide gNA spacers were employed to target the genome, according the sequences in Table 1. FIG. 8 describes the gene targets used in each heterologous genetic circuit cascade. The steps refer to the order in which it is expected the genes were expressed as a consequence of the Cas9-VPR-mediated targeting and activation.

TABLE 1
Spacer Sequences

| SEQ ID NO | Spacer Sequence | Spacer Gene Target |
|---|---|---|
| 1 | AAATGCACCGCTGA | BMI1 |
| 2 | CGCCCTGCTCCGCG | BMI1 |
| 3 | TGTAGTCAGGCCCG | BMI1 |
| 4 | CACGTGACCCGCTg | BMI1 |
| 5 | AGTGTGTCACACGG | ERG |
| 6 | GCATGGCTTCACAC | ERG |
| 7 | GATGATATGCAGCC | ERG |
| 8 | CAGCCCAAAGAAAC | ERG |
| 9 | AGCGACCCCGAGTT | ERG |
| 10 | GGGACCTCGGTCGT | ERG |
| 11 | CTCATCAGCATCGA | ERG |
| 12 | AGTCCGGCCCCAGC | ERG |
| 13 | GGGCGATGTCCGCT | ETS1 |
| 14 | GAAACGCCCTAAAg | ETS1 |
| 15 | CGGCCCAAAGCGAA | ETS1 |
| 16 | GGAGGAGCAGTGCG | ETS1 |
| 17 | GAGCAACACCAGCT | ETV2 |
| 18 | GCTCCAAGAGTCCC | ETV2 |
| 19 | GAACTGCGTCCCCG | ETV2 |
| 20 | GTACAGTCCGTGCG | ETV2 |
| 21 | GGTCCCATGCTTGT | GATA1 |
| 22 | CTCAGTGTGATCCC | GATA1 |
| 23 | AGACGCACATACAC | GATA1 |
| 24 | TCCAAGAATCCCCA | GATA1 |
| 25 | ccgcgggTCAGTCC | GATA2 |
| 26 | GCGCCCCACCACTA | GATA2 |
| 27 | GGAGGGAACGGTCT | GATA2 |
| 28 | GCCCAGAGCATCGA | GATA2 |
| 29 | GCGTTGCGGCCGGT | GATA2 |
| 30 | CCCTGAGTGCCTAG | GATA2 |
| 31 | GTGCGCTCCGGAGA | GATA2 |
| 32 | GGGTGCAGAGCCCC | GATA2 |
| 33 | CTTCGCTAGGAAAG | GFI1 |
| 34 | CTGTAGTTCCCATC | GFI1 |
| 35 | ATGCCCAGTGGTCG | GFI1 |
| 36 | CTGGCTGTGCTTTA | GFI1 |
| 37 | ACTCAGTAACGCTC | GFI1 |
| 38 | CTGTGGGTCGGCAG | GFI1 |
| 39 | GGGACGGGCTGGAA | GFI1 |
| 40 | CAACCCCCGTCGGG | GFI1 |
| 41 | CCGGGGTCGAATTG | HOXA5 |
| 42 | ACTAATAGGGGAGT | HOXA5 |
| 43 | GCGGCAACTggcgg | HOXA5 |
| 44 | GCAGGATTTACGAC | HOXA5 |
| 45 | CCGCCTAGAATGGA | HOXA7 |
| 46 | GCCAACCTCCGCAC | HOXA7 |
| 47 | CTAGCAGGAGTCGG | HOXA7 |
| 48 | GCAGGCTGTTGCGG | HOXA7 |
| 49 | CCGCGCCTTCTTGA | HOXA9 |
| 50 | CCGCACGCTATTAA | HOXA9 |
| 51 | TGCCGCTCTACGAT | HOXA9 |
| 52 | GCACGTGACGCGCA | HOXA9 |
| 53 | AGGTTGCGACTGGA | HOXB4 |
| 54 | GAGAGAGCGAGGCA | HOXB4 |
| 55 | CAGGAGGGCTACAC | HOXB4 |
| 56 | GCGATTCCCGGATA | HOXB4 |
| 57 | CGCGGCCTTAAAAA | IRF8 |
| 58 | AGCGGCTCCAGAAG | IRF8 |
| 59 | CAGCTTGGCTCGCG | IRF8 |
| 60 | CCGCCGCACTCCCG | IRF8 |
| 61 | GCCGCGTAGAAGGA | LCOR |
| 62 | GCGGGACTGACTTC | LCOR |
| 63 | CCGTGGCCCCGGCG | LCOR |
| 64 | AGGAGCAGTTTCGA | LCOR |
| 65 | GGGTTTATCTCAGG | LCOR |
| 66 | CTGGACAACAGTCA | LCOR |
| 67 | GCAGCCTTCTGATT | LCOR |
| 68 | CCGTCAAGTGTAGA | LCOR |
| 69 | GACCTTCCCGAGAG | LMO2 |
| 70 | GCCGAGGGCAGAGA | LMO2 |
| 71 | CTTGGCTTCTTAAA | LMO2 |
| 72 | TCAAATACAATAGA | LMO2 |
| 73 | CAAAGGTGGGCTCG | MIXL1 |
| 74 | AAGCGATTATTCCC | MIXL1 |
| 75 | ATCCACATCCCCGA | MIXL1 |
| 76 | GCCATGTAAGGCAC | MIXL1 |
| 77 | GCGCCGAATGGGAG | MYB |
| 78 | CCGCGACAGTGAGT | MYB |
| 79 | ACCGCGGAGGCGGC | MYB |
| 80 | CAAACAAAGAGCTT | MYB |
| 81 | AAGGGGTCCGCACG | RUNX1 |
| 82 | CGTACAGTAGCGCG | RUNX1 |
| 83 | GTAGACTTTGCAAG | RUNX1 |
| 84 | GTAAGCCCGGCCGG | RUNX1 |
| 85 | GAACCAATTGAGAT | RUNX1 |
| 86 | GCCAGCCCGTTGTA | RUNX1 |
| 87 | GGACTTAACTCTCC | RUNX1 |
| 88 | TATCAGCCAGGGCG | RUNX1 |
| 89 | TTCTTGCGCGCCCG | SATB1 |
| 90 | AGGGCGCTGTGCGA | SATB1 |
| 91 | tggggattaaaacc | SATB1 |
| 92 | GAGGAAGGCGGCGT | SATB1 |
| 93 | GAGATAGTCCCCTT | SPI1 |
| 94 | AGGGATGACTTTGG | SPI1 |
| 95 | CTTGCGCTACATAC | SPI1 |
| 96 | CTCTGCCACACCAC | SPI1 |
| 97 | CGAGGTCACGGCGT | TBX6 |
| 98 | CCGCGTGGTCCGTC | TBX6 |
| 99 | GGGGCAGCGTCCCA | TBX6 |
| 100 | CCAGACCTTCTGGC | TBX6 |
| 101 | GCTTTCACGGAAGG | TBXT |
| 102 | CTGCGCATCGGGTG | TBXT |
| 103 | ccgcACTTCCGCGA | TBXT |
| 104 | GCGCCAAGCGGTGT | TBXT |
| 105 | CAAAGCGAGTTTCC | TAL1 |
| 106 | TAGGGAGACGAGAA | TAL1 |
| 107 | GTCCGTGTTGGGAA | TAL1 |
| 108 | CTGGAATTGGGTTC | TAL1 |
| 109 | TTACAGCGCGTCGG | TAL1 |
| 110 | GAGGCGCTTATCGG | TAL1 |
| 111 | TCGAAAGGAACCGA | TAL1 |
| 112 | GCGTACCCCCGTAG | TAL1 |

### Cell Culture

1.8e5 nucleofected cells were cultured per well in 24 well tissue culture plates treated with Anti-adherence rinsing solution (Stemcell Technologies, Cat #07010) plates rotating at 100 RPM under standard normoxia conditions in a humidified 5% CO2 tissue culture incubator, in 500 ul per well. Day 0 media included: StemPro-34 (Gibco, Cat #10639011), 1× Penicillin-Streptomycin (Gibco Cat #15140122), 2 mL Glutamax (Gibco, Cat #35050061), 50 ug/ml Ascorbic Acid (Santa Cruz Biotechnology, Cat #sc-39430), 4 mM 1-Thioglycerol (Sigma-Aldrich, Cat #M1753), 1× Insulin-Transferrin-Selenium (ITS-G) (Gibco, Cat #41400045) and 10 mM Y-27632 (Bio-Techne Cat #1254), 50 ng/ml rhBMP4 (Peprotec, Cat #120-05ET), 50 ng/ml FGF-2 (Peprotech, Cat #3718-FB) and 10 uM CHIR99021 (Bio-Techne, Cat #4423).

On Day 1, cultures underwent a complete media change with StemPro-34 (Gibco, Cat #10639011), 1× Penicillin-Streptomycin (Gibco Cat #15140122), 2 mL Glutamax (Gibco, Cat #35050061), 50 ug/ml Ascorbic Acid (Santa Cruz Biotechnology, Cat #sc-39430), 4 mM 1-Thioglycerol (Sigma-Aldrich, Cat #M1753), 1× Insulin-Transferrin-Selenium (ITS-G) (Gibco, Cat #41400045), 50 ng/ml rhBMP4 (Peprotec, Cat #120-05ET), 50 ng/ml FGF-2 (Peprotech, Cat #3718-FB), and 50 ng/ml VEGF-165 (Peprotech, Cat #100-20). On Day 2, 6 uM SB431542 (Selleckchem, Cat #101762-616) is added to the cultures. On Day 4, cultures underwent a media change with StemPro-34 (Gibco, Cat #10639011), 1× Penicillin-Streptomycin (Gibco Cat #15140122), 2 mL Glutamax (Gibco, Cat #35050061), 50 ug/ml Ascorbic Acid (Santa Cruz Biotechnology, Cat #sc-39430), 4 mM 1-Thioglycerol (Sigma-Aldrich, Cat #M1753), 1× Insulin-Transferrin-Selenium (ITS-G) (Gibco, Cat #41400045), 50 ng/ml FGF-2 (Peprotech, Cat #3718-FB), 50 ng/ml VEGF-165 (Peprotech, Cat #100-20) and 50 ng/ml SCF (Peprotech, Cat #300-07).

### Flow Cytometry

Cultures were analyzed on Days 3 and 5 by flow cytometry on a Penteon (Agilent, Cat #2010284AA). Embryoid bodies were harvested into 96 deep-well plates (Nest, Cat #503162) and disaggregated in 500 ml TrypLE™ Express Enzyme (Gibco, Cat #12605028) at 37 C for 20-30 minutes followed by mechanical disruption by pipetting. TrypLE™ Express Enzyme is quenched with DMEM-F12 (Stemcell Technologies, Cat #36254). Cells were stained for 20 min at room temperature with Zombie Aqua™ Fixable (Biolegend, Cat #423102, 1:2000), washed with 1XDPBS, 2% FBS, 0.02% NaN3, and blocked for 10 min on ice with Human IgG (Lee Biosolutions Cat #340-21). Cells are stained on ice for 30 mins with PDGFR-BV421 BD Bioscienes Cat #562799, 1:400), CD73-BV785 (Biolegend, Cat #344028, 1:400), CD144/VE-Cadherin-PE (Biolegend, Cat #348505, 1:100), CD34-PE-Dazz (Biolegend, Cat #343534, 1:400), CD309-PE-Cy7 (Biolegend, Cat #359912, 1:100), CD43-APC (Biolegend, Cat #343206, 1:800), CD45-AF700 (Biolegend, Cat #304024, 1:200). The cells were washed with 1XDPBS, 2% FBS, 0.02% NaN3. Cells were resuspended in 1XDPBS, 2% FBS, 0.02% NaN3 for analysis. Data was analyzed using FlowJo™ v10.8 Software (BD Life Sciences). Dead cells and doublets were excluded from analysis via Zombie Aqua and doublet discrimination gates.

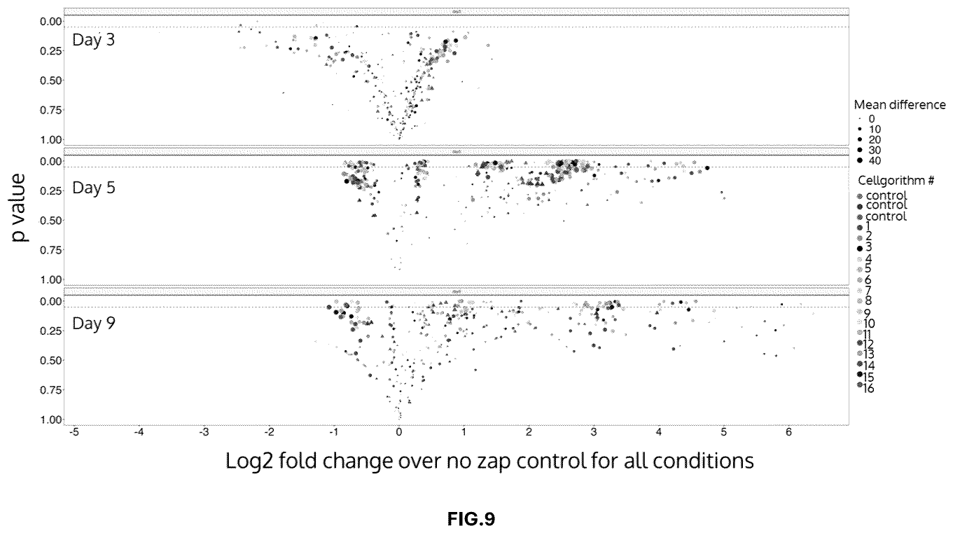

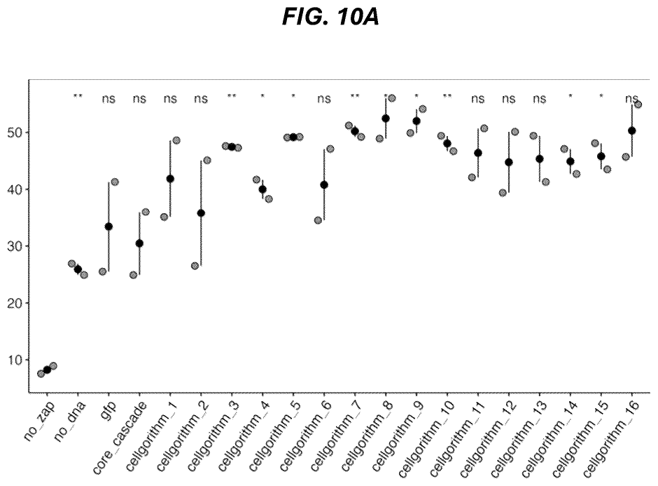

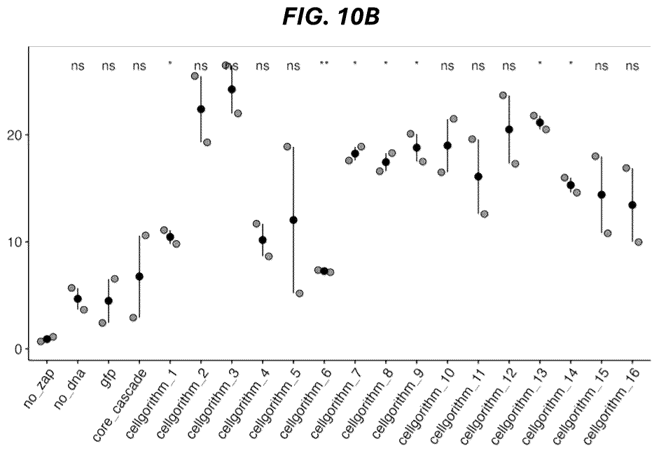

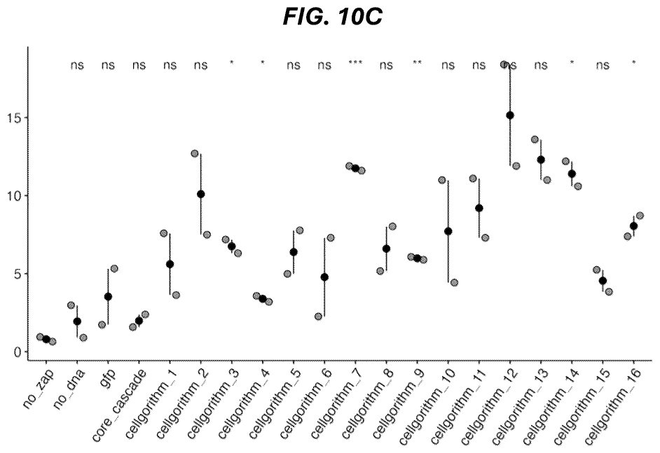

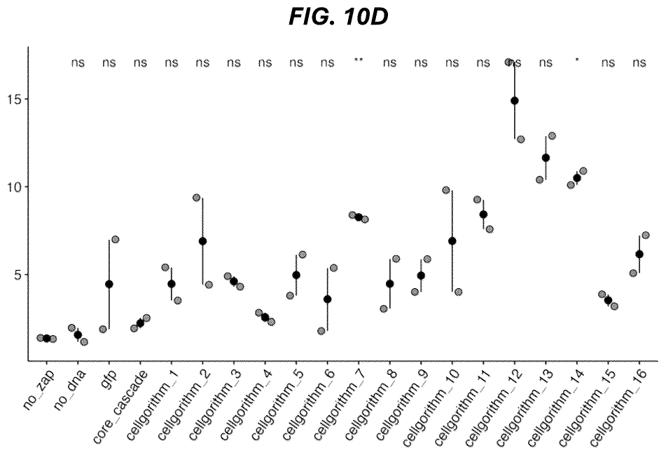

Flow cytometry data was aggregated to form FIG. 10A-10D, which depict the relative frequency of cells at day 5 of culture among appropriately sized, live, singlet cells. Scatter plots were made at varying time points to visualize the magnitude of increased hemogenic gene expression induced by the HGCs over the controls (FIG. 9).

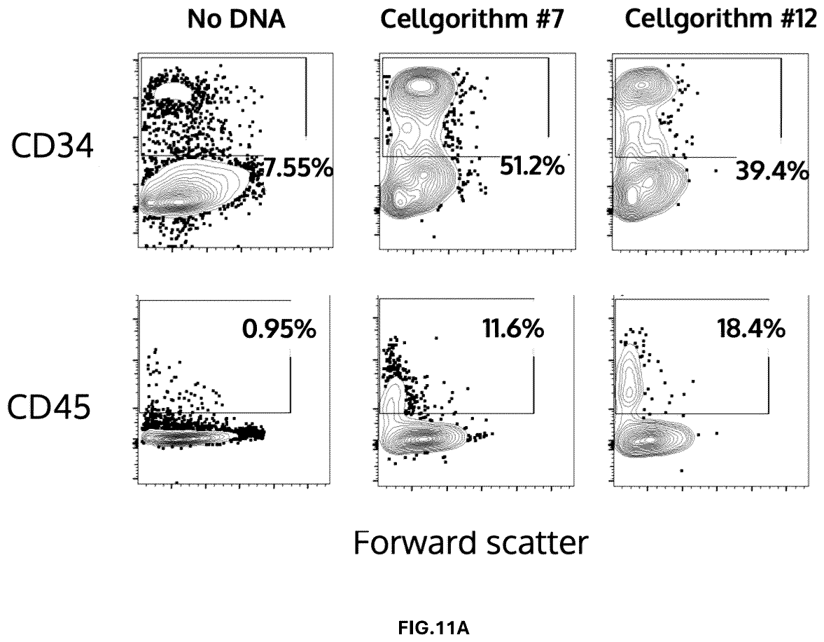

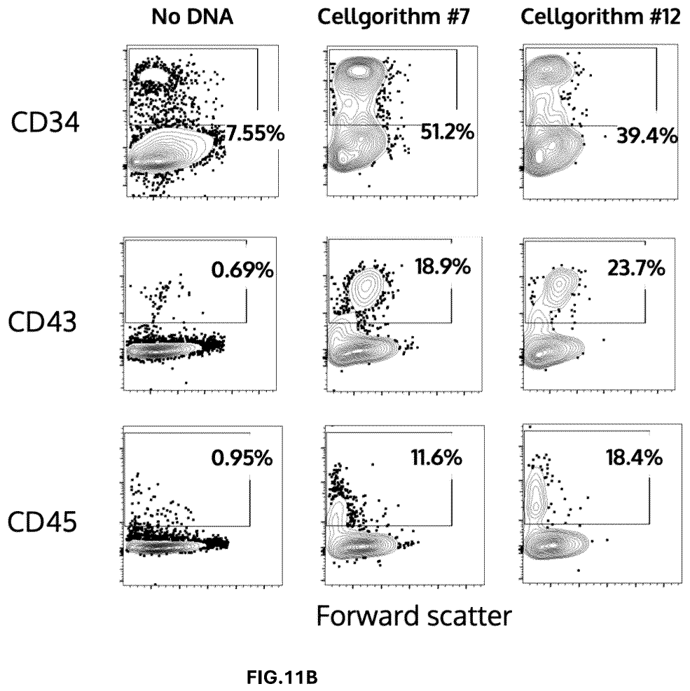

Exemplary results of flow cytometry analysis depicting the frequency of cells expressing the indicated surface marker at day 5 of culture in live singlet cells as compared to the no DNA control (which underwent nucleofection in the absence of exogenous DNA) for HSCs (Cellgorithms) #7 and #12 are shown in FIGS. 11A-11B. Summary graphs shown in FIG. 11C-11D.

### Embodiments

The following non-limiting embodiments provide illustrative examples of the invention, but do not limit the scope of the invention.

Embodiment 1. A method for inducing a desired expression and/or activity profile of a target gene in a cell, the method comprising:
contacting the cell with a heterologous genetic circuit comprising a plurality of gate units, wherein, upon activation of the heterologous genetic circuit, the plurality of gate units operates in concert to induce a plurality of distinct modulations of the target gene in a sequential manner, each of the plurality of distinct modulations being necessary but individually insufficient to effect the desired expression and/or activity profile of the target gene, wherein the plurality of gate units comprises:
   (i) a first gate unit that is activatable, upon the activation of the heterologous genetic circuit, to induce a first distinct modulation of the plurality of distinct modulations; and
   (ii) a second gate unit that is activatable upon the activation of the heterologous genetic circuit, to induce a second distinct modulation of the plurality of distinct modulations, wherein the second distinct modulation is induced subsequent to the first distinct modulation, such that the first distinct modulation and the second distinct modulation both enhance or both reduce expression and/or activity level of the target gene in the cell,
wherein, upon the contacting, the plurality of gate units operates in concert to effect the desired expression and/or activity profile of the target gene in the cell,
optionally wherein:
(1) the method further comprises contacting the cell with an activating moiety to activate the heterologous genetic circuit; and/or
(2) (i) the first gate unit comprises a first gene regulating moiety that is activated upon activation of the first gate unit, to induce the first distinct modulation via specific binding of the first gene regulating moiety to the target gene, and
   (ii) the second gate unit comprises a second gene regulating moiety that is activated upon activation of the second gate unit, to induce the second distinct modulation via specific binding of the first gene regulating moiety to the target gene; and/or
(3) the first gene regulating moiety and the second gene regulating moiety exhibit complementarity to substantially the same polynucleotide sequence of the target gene; and/or
(4) the first gene regulating moiety and the second gene regulating moiety exhibit complementarity to different polynucleotide sequences of the target gene; and/or
(5) the second gate unit further comprises a second gate moiety that is activated upon the activation of the second unit, to induce activation of the second gene regulating moiety via specific binding of the second gate moiety to the second gene regulating moiety,
   optionally wherein:
   (i) the first gate unit further comprises a first gate moiety that is activated upon the activation of the first gate unit, to induce: (a) activation of the first gene regulating moiety via specific binding of the first gate moiety to the first gene regulating moiety, and (b) the activation of the second gate moiety via specific binding of the first gate moiety to the second gate moiety; and/or
   (ii) the activating moiety is capable of inducing: (a) activation of the first gene regulating moiety via specific binding of the activating moiety to the first gene regulating moiety, and (b) the activation of the second gate moiety; and/or
(6) the first distinct modulation and the second distinct modulation both enhance the expression and/or activity level of the target gene; and/or
(7) the first distinct modulation and the second distinct modulation both reduce the expression and/or activity level of the target gene; and/or
(8) the first gate unit is activatable to modulate expression and/or activity profile of an additional target gene, and wherein the second gate unit is activatable to selectively induce the second distinct modulation of the target gene without modulating the expression and/or activity profile of the additional target gene; and/or
(9) the second gate unit is activatable to modulate expression and/or activity profile of an additional target gene, and wherein the first gate unit is not configured to modulate expression and/or activity profile of the additional target gene.

Embodiment 2. A method for inducing a desired expression and/or activity profile of a target gene in a cell, the method comprising:
contacting the cell with a heterologous genetic circuit comprising a plurality of gate units, wherein, upon activation of the heterologous genetic circuit, the plurality of gate units operates in concert to induce a plurality of distinct modulations of the target gene in a sequential manner, each of the plurality of distinct modulations being necessary but individually insufficient to effect the desired expression and/or activity profile of the target gene, wherein the plurality of gate units comprises:

(i) a first gate unit that is activatable, upon the activation of the heterologous genetic circuit, to induce a first distinct modulation of the plurality of distinct modulations; and
(ii) a second gate unit that is activatable to induce disruption of the first gate unit that has been activated, wherein the inactivation induces a second distinct modulation of the plurality of distinct modulations, wherein the second distinct modulation is induced subsequent to the first distinct modulation, such that the second distinct modulation attenuates the first distinct modulation,
wherein, upon the contacting, the plurality of gate units operates in concert to effect the desired expression and/or activity profile of the target gene in the cell,
optionally wherein:
(1) the method further comprises contacting the cell with an activating moiety to activate the heterologous genetic circuit; and/or
(2) (i) the first gate unit comprises a first gene regulating moiety that is activated upon activation of the first gate unit, to induce the first distinct modulation via specific binding of the first gene regulating moiety to the target gene, and
   (ii) the second gate unit comprises a second gene regulating moiety that is activated upon activation of the second gate unit, to induce the second distinct modulation via specific binding of the first gene regulating moiety to the target gene; and/or
(3) the second gate unit further comprises a second gate moiety that is activated upon the activation of the second unit, to induce activation of the second gene regulating moiety via specific binding of the second gate moiety to the second gene regulating moiety,
   optionally wherein:
   (i) the first gate unit further comprises a first gate moiety that is activated upon the activation of the first gate unit, to induce: (a) activation of the first gene regulating moiety via specific binding of the first gate moiety to the first gene regulating moiety, and (b) the activation of the second gate moiety via specific binding of the first gate moiety to the second gate moiety,
      further optionally wherein:
      (A) upon activation of the second gene regulating moiety, the second gene regulating moiety induces the disruption of the first gate unit via disruption of the first gate moiety and/or the first gene regulating moiety; and/or
      (B) the disruption comprises modifying a polynucleotide sequence encoding the first gate moiety and/or the first gene regulating moiety; and/or
   (ii) the activating moiety is capable of inducing: (a) activation of the first gene regulating moiety via specific binding of the activating moiety to the first gene regulating moiety, and (b) the activation of the second gate moiety,
      further optionally wherein:
      (A) upon activation of the second gene regulating moiety, the second gene regulating moiety induces the disruption of the first gate unit via disruption of the first gene regulating moiety; and/or
      (B) the disruption comprises modifying a polynucleotide sequence encoding the first gene regulating moiety; and/or
(4) the first gate unit is activatable to modulate expression and/or activity profile of an additional target gene, and wherein the second gate unit is activatable to selectively induce the second distinct modulation of the target gene without modulating the expression and/or activity profile of the additional target gene; and/or
(5) the second gate unit is activatable to modulate expression and/or activity profile of an additional target gene, and wherein the first gate unit is not configured to modulate expression and/or activity profile of the additional target gene; and/or
(6) the first gene regulating moiety and the second gene regulating moiety exhibit complementarity to substantially the same polynucleotide sequence of the target gene; and/or
(7) the first gene regulating moiety and the second gene regulating moiety exhibit complementarity to different polynucleotide sequences of the target gene.

Embodiment 3. The method of Embodiment 1 or Embodiment 2, further optionally wherein:
(1) the target gene comprises a plurality of target genes; and/or
(2) the second distinct modulation induces selective modulation of one of the plurality of target genes, but not that of the other of the plurality of target genes; and/or
(3) the activating moiety comprises a guide nucleic acid (gNA) capable of forming a complex with an endonuclease, wherein the gNA is capable of binding to at least a portion of the heterologous genetic circuit to thereby activate the heterologous genetic circuit; and/or
(4) each of the first gate unit, the first gate moiety, the first gene regulating moiety, the second gate unit, the second gate moiety, and/or the second gene regulating moiety comprises a gNA that is activatable,
   wherein, upon activation of the gNA, the gNA forms a complex with an endonuclease; and/or
(5) (i) the activatable gNA comprises a self-cleaving gNA, and/or (ii) the activatable gRN comprises a non-canonical termination sequence; and/or
(6) the endonuclease comprises a CRISPR/Cas protein,
   optionally wherein the CRISPR/Cas protein is operatively coupled to a gene activator or a gene repressor; and/or
(7) induction of the second distinct modulation is configured to occur at least about 5 minutes, at least about 1 hour, at least about 6 hours, or at least about 12 hours subsequent to induction of the first distinct modulation; and/or
(8) the activating moiety consists of a single activating moiety; and/or
(9) upon activation of the second gate unit, the second gate unit is further capable of modulating expression and/or activity of an additional target gene in the cell; and/or
(10) the second gate unit comprises an additional gene regulating moiety that is activated upon activation of the second gate unit, to induce modulation of the expression and/or activity of the additional target gene via specific binding of the additional gene regulating moiety to the additional target gene; and/or
(11) the target gene is endogenous to the cell; and/or
(12) the desired expression and/or activity profile of the target gene induces differentiation of the cell toward a target cell type; and/or
(13) the target cell type exhibits a target phenotype; and/or
(14) the target cell type is a tissue-specific cell, optionally wherein the tissue specific cell is selected from the group consisting of immune cells, neurons, osteoblasts, endothelial cells, mesenchymal cells, and epithelial cells; and/or
(15) the target phenotype comprises (i) expression or activity level of a target protein, (ii) an average cellular dimension, and/or (iii) a desired function; and/or
(16) the target gene encodes a cell differentiation regulatory factor; and/or
(17) the cell differentiation regulatory factor comprises a growth factor; and/or
(18) the cell differentiation regulatory factor comprises a transcription factor; and/or
(19) the cell differentiation regulatory factor comprises an immune cell regulatory factor comprising E2A, Ikaros, Runx1, Lmo2, PU.1, Hes1, Tcf7, Gata3, and Bcl11b; and/or
(20) the cell differentiation regulatory factor comprises a T-box transcription factor (TBX), a basic helix-loop-helix transcription factor (bHLH), a homeobox protein, a forkhead box (FOX), a SRY-related HMG-box (SOX), and/or a GATA protein; and/or
(20) the cell is a stem cell selected from the group consisting of induced pluripotent stem cells (iPSC), embryonic stem cells (ESC), mesenchymal stem cells (MSC), hematopoietic stem cells (HSC), neural stem cells, and epithelial stem cells.

Embodiment 4. A system for inducing a desired expression and/or activity profile of a target gene in a cell, the system comprising:
a heterologous genetic circuit comprising a plurality of gate units, wherein, upon activation of the heterologous genetic circuit, the plurality of gate units operates in concert to induce a plurality of distinct modulations of the target gene in a sequential manner, each of the plurality of distinct modulations being necessary but individually insufficient to effect the desired expression and/or activity profile of the target gene, wherein the plurality of gate units comprises:
   (i) a first gate unit that is activatable, upon the activation of the heterologous genetic circuit, to induce a first distinct modulation of the plurality of distinct modulations; and
   (ii) a second gate unit that is activatable upon the activation of the heterologous genetic circuit, to induce a second distinct modulation of the plurality of distinct modulations, wherein the second distinct modulation is induced subsequent to the first distinct modulation, such that the first distinct modulation and the second distinct modulation both enhance or both reduce expression and/or activity level of the target gene in the cell,
wherein, upon the activation of the heterologous genetic circuit, the plurality of gate units operates in concert to effect the desired expression and/or activity profile of the target gene in the cell,
optionally wherein:
(1) the system further comprises an activating moiety for activating the heterologous genetic circuit; and/or
(2) (i) the first gate unit comprises a first gene regulating moiety that is activated upon activation of the first gate unit, to induce the first distinct modulation via specific binding of the first gene regulating moiety to the target gene, and
   (ii) the second gate unit comprises a second gene regulating moiety that is activated upon activation of the second gate unit, to induce the second distinct modulation via specific binding of the first gene regulating moiety to the target gene; and/or
(3) the first gene regulating moiety and the second gene regulating moiety exhibit complementarity to substantially the same polynucleotide sequence of the target gene; and/or
(4) the first gene regulating moiety and the second gene regulating moiety exhibit complementarity to different polynucleotide sequences of the target gene; and/or
(5) the second gate unit further comprises a second gate moiety that is activated upon the activation of the second unit, to induce activation of the second gene regulating moiety via specific binding of the second gate moiety to the second gene regulating moiety,
   optionally wherein:
   (i) the first gate unit further comprises a first gate moiety that is activated upon the activation of the first gate unit, to induce: (a) activation of the first gene regulating moiety via specific binding of the first gate moiety to the first gene regulating moiety, and (b) the activation of the second gate moiety via specific binding of the first gate moiety to the second gate moiety; and/or
   (ii) the activating moiety is capable of inducing: (a) activation of the first gene regulating moiety via specific binding of the activating moiety to the first gene regulating moiety, and (b) the activation of the second gate moiety; and/or
(6) the first distinct modulation and the second distinct modulation both enhance the expression and/or activity level of the target gene; and/or
(7) the first distinct modulation and the second distinct modulation both reduce the expression and/or activity level of the target gene; and/or
(8) the first gate unit is activatable to modulate expression and/or activity profile of an additional target gene, and wherein the second gate unit is activatable to selectively induce the second distinct modulation of the target gene without modulating the expression and/or activity profile of the additional target gene; and/or
(9) the second gate unit is activatable to modulate expression and/or activity profile of an additional target gene, and wherein the first gate unit is not configured to modulate expression and/or activity profile of the additional target gene.

Embodiment 5. A system for inducing a desired expression and/or activity profile of a target gene in a cell, the system comprising:
a heterologous genetic circuit comprising a plurality of gate units, wherein, upon activation of the heterologous genetic circuit, the plurality of gate units operates in concert to induce a plurality of distinct modulations of the target gene in a sequential manner, each of the plurality of distinct modulations being necessary but individually insufficient to effect the desired expression and/or activity profile of the target gene, wherein the plurality of gate units comprises:
   (i) a first gate unit that is activatable, upon the activation of the heterologous genetic circuit, to induce a first distinct modulation of the plurality of distinct modulations; and
   (ii) a second gate unit that is activatable to induce disruption of the first gate unit that has been activated, wherein the inactivation induces a second distinct modulation of the plurality of distinct modulations, wherein the second distinct modulation is induced subsequent to the first distinct modulation, such that the second distinct modulation attenuates the first distinct modulation,
wherein, upon the activation of the heterologous genetic circuit, the plurality of gate units operates in concert to effect the desired expression and/or activity profile of the target gene in the cell,
optionally wherein:
(1) the system further comprises an activating moiety for activating the heterologous genetic circuit; and/or
(2) (i) the first gate unit comprises a first gene regulating moiety that is activated upon activation of the first gate unit, to induce the first distinct modulation via specific binding of the first gene regulating moiety to the target gene, and
   (ii) the second gate unit comprises a second gene regulating moiety that is activated upon activation of the second gate unit, to induce the second distinct modulation via specific binding of the first gene regulating moiety to the target gene; and/or
(3) the second gate unit further comprises a second gate moiety that is activated upon the activation of the second unit, to induce activation of the second gene regulating moiety via specific binding of the second gate moiety to the second gene regulating moiety,
   optionally wherein:
   (i) the first gate unit further comprises a first gate moiety that is activated upon the activation of the first gate unit, to induce: (a) activation of the first gene regulating moiety via specific binding of the first gate moiety to the first gene regulating moiety, and (b) the activation of the second gate moiety via specific binding of the first gate moiety to the second gate moiety,
      further optionally wherein:
      (A) upon activation of the second gene regulating moiety, the second gene regulating moiety induces the disruption of the first gate unit via disruption of the first gate moiety and/or the first gene regulating moiety; and/or
      (B) the disruption comprises modifying a polynucleotide sequence encoding the first gate moiety and/or the first gene regulating moiety; and/or
   (ii) the activating moiety is capable of inducing: (a) activation of the first gene regulating moiety via specific binding of the activating moiety to the first gene regulating moiety, and (b) the activation of the second gate moiety,
      further optionally wherein:
      (A) upon activation of the second gene regulating moiety, the second gene regulating moiety induces the disruption of the first gate unit via disruption of the first gene regulating moiety; and/or
      (B) the disruption comprises modifying a polynucleotide sequence encoding the first gene regulating moiety; and/or
(4) the first gate unit is activatable to modulate expression and/or activity profile of an additional target gene, and wherein the second gate unit is activatable to selectively induce the second distinct modulation of the target gene without modulating the expression and/or activity profile of the additional target gene; and/or
(5) the second gate unit is activatable to modulate expression and/or activity profile of an additional target gene, and wherein the first gate unit is not configured to modulate expression and/or activity profile of the additional target gene; and/or
(6) the first gene regulating moiety and the second gene regulating moiety exhibit complementarity to substantially the same polynucleotide sequence of the target gene; and/or
(7) the first gene regulating moiety and the second gene regulating moiety exhibit complementarity to different polynucleotide sequences of the target gene.

Embodiment 6. The system of Embodiment 4 or Embodiment 5, further optionally wherein:
(1) the target gene comprises a plurality of target genes; and/or
(2) the second distinct modulation induces selective modulation of one of the plurality of target genes, but not that of the other of the plurality of target genes; and/or
(3) the activating moiety comprises a guide nucleic acid (gNA) capable of forming a complex with an endonuclease, wherein the gNA is capable of binding to at least a portion of the heterologous genetic circuit to thereby activate the heterologous genetic circuit; and/or
(4) each of the first gate unit, the first gate moiety, the first gene regulating moiety, the second gate unit, the second gate moiety, and/or the second gene regulating moiety comprises a gNA that is activatable,
   wherein, upon activation of the gNA, the gNA forms a complex with an endonuclease; and/or
(5) (i) the activatable gNA comprises a self-cleaving gNA, and/or (ii) the activatable gRN comprises a non-canonical termination sequence; and/or
(6) the endonuclease comprises a CRISPR/Cas protein,
   optionally wherein the CRISPR/Cas protein is operatively coupled to a gene activator or a gene repressor; and/or
(7) induction of the second distinct modulation is configured to occur at least about 5 minutes, at least about 1 hour, at least about 6 hours, or at least about 12 hours subsequent to induction of the first distinct modulation; and/or
(8) the activating moiety consists of a single activating moiety; and/or
(9) upon activation of the second gate unit, the second gate unit is further capable of modulating expression and/or activity of an additional target gene in the cell; and/or
(10) the second gate unit comprises an additional gene regulating moiety that is activated upon activation of the second gate unit, to induce modulation of the expression and/or activity of the additional target gene via specific binding of the additional gene regulating moiety to the additional target gene; and/or
(11) the target gene is endogenous to the cell; and/or
(12) the desired expression and/or activity profile of the target gene induces differentiation of the cell toward a target cell type; and/or
(13) the target cell type exhibits a target phenotype; and/or
(14) the target cell type is a tissue-specific cell, optionally wherein the tissue specific cell is selected from the group consisting of immune cells, neurons, osteoblasts, endothelial cells, mesenchymal cells, and epithelial cells; and/or
(15) the target phenotype comprises (i) expression or activity level of a target protein, (ii) an average cellular dimension, and/or (iii) a desired function; and/or
(16) the target gene encodes a cell differentiation regulatory factor; and/or
(17) the cell differentiation regulatory factor comprises a growth factor; and/or
(18) the cell differentiation regulatory factor comprises a transcription factor; and/or
(19) the cell differentiation regulatory factor comprises an immune cell regulatory factor comprising E2A, Ikaros, Runx1, Lmo2, PU.1, Hes1, Tcf7, Gata3, and Bcl11b; and/or
(20) the cell differentiation regulatory factor comprises a T-box transcription factor (TBX), a basic helix-loop-helix transcription factor (bHLH), a homeobox protein, a forkhead box (FOX), a SRY-related HMG-box (SOX), and/or a GATA protein; and/or (20) the cell is a stem cell selected from the group consisting of induced pluripotent stem cells (iPSC), embryonic stem cells (ESC), mesenchymal stem cells (MSC), hematopoietic stem cells (HSC), neural stem cells, and epithelial stem cells.

Embodiment 7. An engineered cell comprising the system of any one Embodiments 4-6.

Embodiment 8. A composition comprising the system of any one Embodiments 4-6 or the engineered cell of Embodiment 7,
optionally wherein:
(1) the composition further comprises a separate therapeutic agent,
   further optionally wherein the separate therapeutic agent comprises a chemotherapeutic agent, an immunosuppressing agent, and/or an antibiotic agent; and/or
(2) the composition further comprises the activating moiety; and/or
(3) the composition comprises the engineered cell, and wherein the composition is substantially free of the activating moiety.

Systems and methods of the present disclosure may be combined with or modified by other systems and methods, such as, for example, those described in International Patent Application No. PCT/US2018/052211, which is incorporated herein by reference in its entirety.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for inducing a desired expression and/or activity profile of a target gene in a cell, the method comprising:
contacting the cell with a heterologous genetic circuit comprising a plurality of gate units, wherein, upon activation of the heterologous genetic circuit, the plurality of gate units operates in concert to induce a plurality of distinct modulations of the target gene in a sequential manner, each of the plurality of distinct modulations being necessary but individually insufficient to effect the desired expression and/or activity profile of the target gene, wherein the plurality of gate units comprises:
(i) a first gate unit that is activatable, upon the activation of the heterologous genetic circuit, to induce a first distinct modulation of the plurality of distinct modulations; and
(ii) a second gate unit that is activatable upon the activation of the heterologous genetic circuit, to induce a second distinct modulation of the plurality of distinct modulations, wherein the second distinct modulation is induced subsequent to the first distinct modulation, such that the first distinct modulation and the second distinct modulation both enhance or both reduce expression and/or activity level of the target gene in the cell,
wherein, upon the contacting, the plurality of gate units operates in concert to effect the desired expression and/or activity profile of the target gene in the cell.

2. The method of claim 1, further comprising contacting the cell with an activating moiety to activate the heterologous genetic circuit.

3. The method of claim 2, wherein the activating moiety consists of a single activating moiety.

4. The method of claim 2, wherein the activating moiety comprises a guide nucleic acid (gNA) capable of forming a complex with an endonuclease, wherein the gNA is capable of binding to at least a portion of the heterologous genetic circuit to thereby activate the heterologous genetic circuit.

5. The method of claim 1, wherein the first distinct modulation and the second distinct modulation both enhance the expression and/or activity level of the target gene.

6. (canceled)

7. The method of claim 1, wherein the first gate unit is activatable to modulate expression and/or activity profile of an additional target gene, and wherein the second gate unit is activatable to selectively induce the second distinct modulation of the target gene without modulating the expression and/or activity profile of the additional target gene.

8. (canceled)

9. The method of claim 1, wherein the target gene comprises a plurality of target genes.

10. The method of claim 1, wherein induction of the second distinct modulation is configured to occur at least about 5 minutes, at least about 1 hour, at least about 6 hours, or at least about 12 hours subsequent to induction of the first distinct modulation.

11. The method of claim 1, wherein the target gene is endogenous to the cell.

12. The method of claim 1, wherein the target gene encodes a cell differentiation regulatory factor.

13. (canceled)

14. The method of claim 1, the desired expression and/or activity profile of the target gene induces differentiation of the cell toward a target cell type.

15. The method of claim 1, wherein the cell is a stem cell.

16. (canceled)

17. The method of claim 1, wherein:
(i) the first gate unit comprises a first gene regulating moiety that is activated upon activation of the first gate unit, to induce the first distinct modulation via specific binding of the first gene regulating moiety to the target gene, and
(ii) the second gate unit comprises a second gene regulating moiety that is activated upon activation of the second gate unit, to induce the second distinct modulation via specific binding of the first gene regulating moiety to the target gene.

18. The method of claim 17, wherein the first gene regulating moiety and the second gene regulating moiety exhibit complementarity to substantially the same polynucleotide sequence of the target gene.

19. The method of claim 17, wherein the first gene regulating moiety and the second gene regulating moiety exhibit complementarity to different polynucleotide sequences of the target gene.

20. The method of claim 17, wherein the second gate unit further comprises a second gate moiety that is activated upon the activation of the second unit, to induce activation of the second gene regulating moiety via specific binding of the second gate moiety to the second gene regulating moiety.

21. The method of claim 20, wherein the first gate unit further comprises a first gate moiety that is activated upon the activation of the first gate unit, to induce:
(a) activation of the first gene regulating moiety via specific binding of the first gate moiety to the first gene regulating moiety, and
(b) the activation of the second gate moiety via specific binding of the first gate moiety to the second gate moiety.

22. The method of claim 20, wherein the activating moiety is capable of inducing:
(a) activation of the first gene regulating moiety via specific binding of the activating moiety to the first gene regulating moiety, and
(b) the activation of the second gate moiety.

23. The method of claim 21, wherein each of the first gate unit, the first gate moiety, the first gene regulating moiety, the second gate, the second gate moiety, and/or the second gene regulating moiety comprises a gNA that is activatable,
wherein, upon activation of the gNA, the gNA forms a complex with an endonuclease.

24. The method of claim 23, wherein the activatable gNA comprises a non-canonical termination sequence.

25-100. (canceled)
